(19) United States
(12) Patent Application Publication
(10) Pub. No.: US 2026/0345371 A1
(43) Pub. Date: Nov. 5, 2026

(54) FLUID COLLECTION DEVICE HOLDERS, AND RELATED SYSTEMS AND METHODS

(71) Applicant: PUREWICK CORPORATION, Covington, GA (US)

(72) Inventors: Raymond J. Newton, Bonsall, CA (US); Camille Newton, Bonsall, CA (US); Serena Agrawal, Atlanta, GA (US)

(21) Appl. No.: 19/474,429

(22) PCT Filed: Apr. 13, 2023
(86) PCT No.: PCT/US2023/018474

§ 371 Date: Oct. 10, 2025

(51) Int. Cl. A61F 5/455; A61F 5/453

(52) U.S. Cl. CPC A61F 5/455; A61F 5/453

(57) ABSTRACT

Embodiments disclosed herein are fluid collection assemblies including a fluid collection device and a holder. The holder is configured to selectively and detachably secure to the fluid collection device. The holder includes a first region configured to be disposed at least proximate to a supra-pubic region of the user and a second region adjustable relative to the first region and configured to hold the opening of the fluid collection device at least proximate to the urethra of the user when the first region is disposed at least proximate to the supra-pubic region of the user.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, can be used to address some of these circumstances. However, bed pans and urinary catheters have several problems associated therewith. For example, bed pans can be prone to discomfort, pressure ulcers spills, and other hygiene issues. Urinary catheters be can be uncomfortable, painful, and can cause urinary tract infections.

Thus, users and manufacturers of fluid collection devices continue to seek new and improved devices, systems, and methods to collect urine.

### SUMMARY

Embodiments disclosed herein include holders for fluid collection devices, and related assemblies, systems, and methods of use. In an embodiment, a fluid collection assembly includes a fluid collection device and a holder. The fluid collection device includes a fluid impermeable barrier at least partially defining a chamber. The fluid impermeable barrier includes an opening in fluid communication with the chamber, and an aperture substantially distal to the opening and in fluid communication with the chamber. The opening is configured to be positioned at least proximate to or receive therein a urethra of a user. The holder is secured or securable to the fluid collection device, and includes a first region and a second region. The first region is configured to be disposed at least proximate to a supra-pubic region of the user or between the legs of the user. The second region is adjustable relative to the first region and configured to hold the opening of the fluid collection device at least proximate to the urethra of the user when the first region is disposed at least proximate to the supra-pubic region of the user or against the user with the urethra received therein when the first region is disposed between the legs of the user.

In an embodiment, a fluid collection device that include a holder body and a shape memory material. The holder body is elongated and includes a first region and a second region. The shape memory material extends longitudinally along the holder body at least partially in the first region and at least partially in the second region of the holder body. The shape memory material is configured to bend and/or contour the holder body to a substantially L-shaped configuration effective to dispose the first region of the holder body at least proximate to a supra-pubic region of the user while the second region of the holder body holds an opening of the fluid collection device at least proximate to the urethra of the user.

In an embodiment, a method of collecting fluid from a user. The method includes providing a fluid collection device including a fluid impermeable barrier defining a chamber, an opening having fluid communication with the chamber and configured to be placed proximate to a urethra of the user, and an aperture configured to receive a conduit therethrough to provide fluid communication between the chamber and the conduit. The method includes securing the fluid collection device to a holder. The method includes bending the holder such that a first region of the holder is disposed at least proximate to a supra-pubic region of the user and a second region of the holder holds the opening of the fluid collection device at least proximate to the urethra of the user. The method includes collecting fluid in the chamber of the fluid collection device.

In an embodiment, a method of collecting fluid from a user. The method includes providing a fluid collection device including a sheath having a fluid impermeable barrier having a proximal region and a distal region extending from the proximal region, an opening in the proximal region, and an aperture in the distal region. The fluid impermeable barrier at least partially defines a chamber in fluid communication with the opening and the aperture. The method includes inserting a penis of the user through the opening at least partially into the chamber. The method includes securing a base of the fluid collection device to the user at least proximate to the penis. The method includes bending a first region of a holder secured to the fluid collection device such that the first region of the holder conforms to a perineum of the user between the legs of the user. The holder includes a second region secured to the fluid collection device at least proximate to the opening.

In an embodiment, a holder for a fluid collection device includes a first region and a second region. The first region is elongated and includes a shape memory material configured to bend to conform to a perineum between legs of a user. The second region extends from the first region and is configured to secure to a fluid collection device proximate to an opening in the fluid collection device for insertion of the penis. The second region is adjustable relative to the first region and, when secured to the fluid collection device, is configured to hold the opening of the fluid collection device against the user when the penis of the user is inserted into the opening.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection device, according to an embodiment.

FIG. 1B is a front view of a female user wearing the fluid collection device of FIG. 1A.

FIG. 1C is an exploded isometric view of the female fluid collection device of FIG. 1A.

FIG. 1D is a cross-sectional view of the female fluid collection device of FIG. 1A taken along line 1-1 thereof, according to various embodiments.

FIG. 1E is a block diagram of a system for fluid collection, according to an embodiment.

FIG. 2A is a front view of a holder in a partially bent position, according to an embodiment.

FIG. 2B is an isometric side view of the holder of FIG. 2A in a bent position, according to an embodiment.

FIG. 2C is an isometric side view of a fluid collection assembly including the holder of FIG. 2A in a bent position with a fluid collection device secured thereto, according to an embodiment.

FIG. 3A is a front view of a holder in a partially bent position and a fluid collection device, according to an embodiment.

FIG. 3B is an isometric side view of a fluid collection assembly including the holder of FIG. 3A in a bent position with a fluid collection device secured thereto, according to an embodiment.

FIG. 3C is a front view of a holder in a partially bent position, according to an embodiment.

FIG. 4 is an isometric view of fluid collection device and the holder of FIG. 2A in use on a user, according to an embodiment.

FIG. 5 is a flow diagram of a method to collect fluid, according to an embodiment.

FIG. 6A is an isometric side view of a holder of a fluid collection assembly in a bent position, according to an embodiment.

FIG. 6B is an isometric side view of a fluid collection device secured to the holder of FIG. 6A, according to an embodiment.

FIG. 6C is an isometric side view of a holder of a fluid collection assembly in a bent position, according to an embodiment.

FIG. 6D is an isometric side view of a fluid collection device secured to the holder of FIG. 6C, according to an embodiment.

FIG. 6E is an isometric side view of a holder of a fluid collection assembly in a bent position, according to an embodiment.

FIG. 6F is an isometric side view of a fluid collection device secured to the holder of FIG. 6E, according to an embodiment.

FIG. 7A is a partial disassembled view of a fluid collection assembly, according to an embodiment.

FIG. 7B is an isometric view of the fluid collection assembly of FIG. 7A with a first region of the holder bent.

FIG. 7C is a front isometric view of the fluid collection assembly of FIG. 7A during use.

FIG. 7D is an isometric view of the fluid collection assembly of FIG. 7A during use with the fluid collection device lifted such that some of the holder is visible.

### DETAILED DESCRIPTION

Embodiments disclosed herein include holders for fluid collection devices, and related assemblies, systems, and methods of use. Fluid collection devices are used to collect fluids such as urine, vaginal discharge, penile discharge, reproductive fluids, blood, sweat, or other bodily fluids from a user. Depending the physical characteristics of a user, rluid collection devices can sometimes be difficult to maintain in a position at least proximate to a urethra of a user. When a fluid collection device is moved during use and/or improperly positioned, the fluid collection device may not effectively collected fluid from the user. For example, when a fluid collection device is moved during use and/or improperly positioned, urine discharged by a user may flow or leak elsewhere on the user or the clothes of the user rather than into the fluid collection device.

At least one, some, or all embodiments of holders disclosed herein are configured to maintain the correct and effective position of the fluid collection device during use, and prevent the fluid collection device from falling away from the anatomy of the user. The correct and effective position of the fluid collection device may include having the urethra of the user disposed about ⅓ of the distance from the top of the opening in the fluid collection device, and the bottom of the fluid collection device tucked against the perineum in the anal region of the user. At least one, some, or all embodiments of holders disclosed herein, then, result in the technical effect of more effectively collecting fluid from a user, preventing undesirable and embarrassing leaks of fluid, and/or improving the sanitary conditions of collected fluid from a user by ensuring fluid is directed into the fluid collection device. In at least one, some, or all embodiments, a proximal or first region of the holder secures to or presses against a supra-pubic region of a user. As used herein, the supra-pubic region may include a region of the user between the navel and genital organs of the user. In at least one, some, or all embodiments, a distal or second region of the holder pressed the fluid collection device towards or against the urethra of the user. Embodiments of holders disclosed herein may mimic a user using their hand to press the fluid collection device against the anatomy of the user. In some embodiments, the distal or second region of the holder is detachably and selectively secured to a distal end of the fluid collection device.

In use, at least one, some, or all embodiments of the holder may be secured to the fluid collection device, and the fluid collection device may positioned on the user with an opening of the fluid collection device at least proximate to a urethra of the user or otherwise positioned to receive fluid discharged from the user. A user or caregiver may then bend the holder to conform or contour to the body of the user. More specifically, a user or caregiver may bend the holder such that the proximal or first region of the holder is pressed against or interfaces the supra-pubic region of the user while the distal or second region of the holder holds, presses, or biases the fluid collection device against or towards the urethra of the user. In many embodiments, this bending results in the holder taking a substantially L-shaped configuration. Adhesive tape may be used to secure the proximal or first region of the holder to the skin of the user at the supra-pubic region. The adhesive tape may be easily removable and replaceable, such as silicone dot adhesive tape. In some embodiments, an elbow connector may be used to connect a conduit of the fluid collection device to an additional conduit in fluid communication with a vacuum source.

At least one, some, or all embodiments of the holders disclosed herein result in the technical effect of decreasing the cost of manufacturing adhesive strips directly on the fluid impermeable barrier of the fluid collection device. At least one, some, or all embodiments or holders disclosed herein result in the technical effect of simpler manufacturing than conventional systems for retaining a fluid collection device in place. At least one, some, or all embodiments of the holders disclosed herein result in the technical effect of more simple and efficient changing of soiled or defective fluid collection devices. For example, during use, the holder may not even need to be removed in order for a user or caregiver to change the fluid collection device being used. At least one, some, or all embodiments of holders disclosed herein also result in the technical effect of providing a lightweight, comfortable, flexible, and/or reusable holder that conforms to the body of individual users.

Embodiments of holders disclosed herein may be used in an assembly including a fluid collection device and the holder. Fluid collection devices used with the holders disclosed herein may be shaped and sized to be positioned adjacent to the opening of a female urethra, positioned over a male penis, receive at least a portion of a penis, or otherwise used for fluid collection from male or females. Although drawings illustrate embodiments used with fluid collection devices on a female or a male, fluid collection devices configured for use with a male or female may be used with fluid collection assemblies disclosed herein. In some embodiments, the fluid collection device may include a fluid impermeable barrier at least partially defining a chamber (e.g., interior region of the fluid collection device) of the fluid collection device. The fluid impermeable barrier also defines an opening extending therethrough from the external environment. The opening may be positioned adjacent to a female urethra or have a male urethra positioned therethrough. The fluid collection device may include a fluid permeable body disposed within the fluid impermeable barrier. The conduit may extend into the fluid collection device at a first end region, through one or more of the fluid impermeable barrier, fluid permeable body to a second end region of the fluid collection device. Exemplary fluid collection devices for use with the systems and methods herein are described in more detail below.

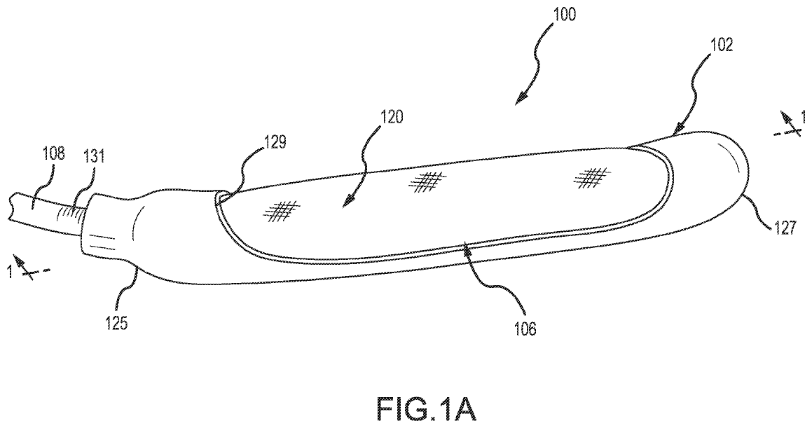

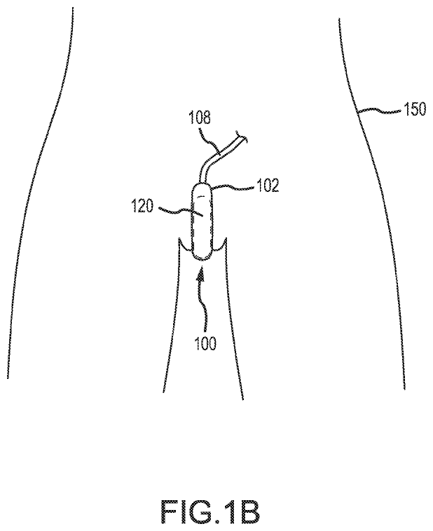

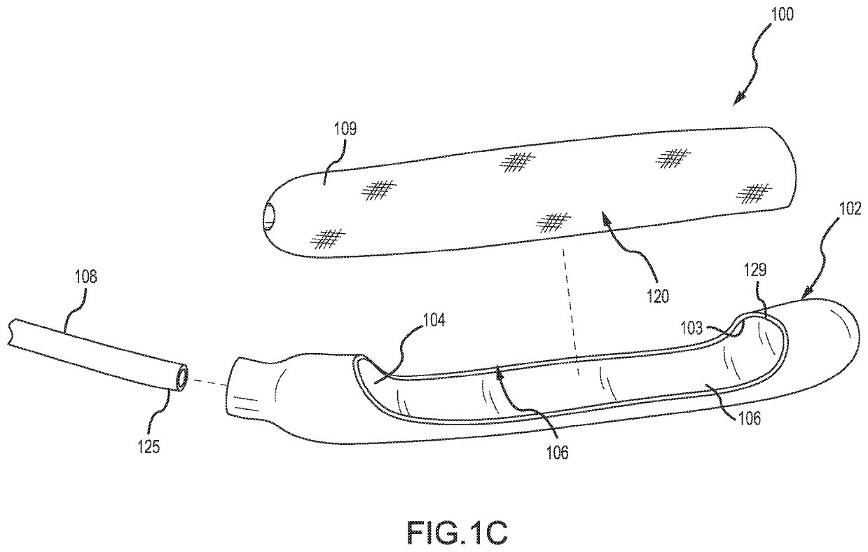

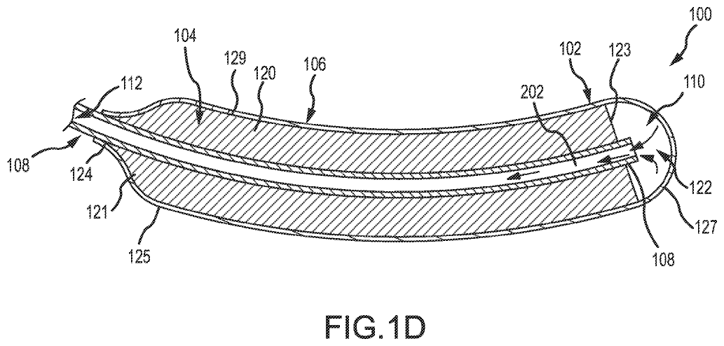

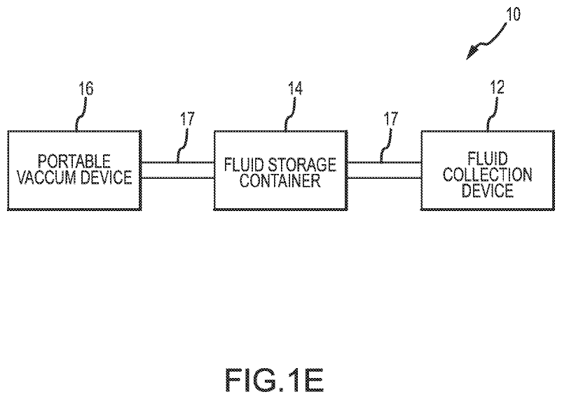

FIGS. 1A-1D show a fluid collection device 100 that may be used with any of the holders disclosed herein, according to some embodiments. Holders disclosed herein, however, are not limited to use with the fluid collection device 100 shown in FIGS. 1A-1D, and may be used with other fluid collection devices. FIG. 1A is an isometric view of a fluid collection device 100, according to an embodiment. The fluid collection device 100 is an example of a female fluid collection device 100 that is configured to receive fluids from a female. The fluid collection device 100 includes a fluid impermeable barrier 102 having a first end region 125 and a second end region 127. The fluid impermeable barrier 102 at least partially defines a chamber 104 (e.g., interior region, shown in FIG. 1C) and includes an inward border or edge 129 defining an opening 106. The fluid impermeable barrier 102 is substantially cylindrical in shape between the first end region 125 and the second end region 127. In other embodiments, the fluid impermeable barrier 102 may include other shapes, such as one of more substantially planar surfaces, triangular, or other suitable shape. The opening 106 is formed in and extends longitudinally through the fluid impermeable barrier 102, thereby enabling fluids to enter the chamber 104 from outside of the fluid collection device 100. The opening 106 may be configured to be positioned at least proximate (e.g., adjacent to, interfacing, or contacting) the opening of a female urethra or over a penis of a male.

With the fluid collection device 100 may positioned at least proximate to the opening of the female urethra or over a penis, urine may enter the interior region of the fluid collection device 100 via the opening 106. The fluid collection device 100 is configured to receive the fluids into the chamber 104 via the opening 106. For example, the opening 106 may exhibit an elongated shape that is configured to extend from a first location below the urethral opening (e.g., the perineum, at or near the anus, or below the vaginal opening) to a second location above the urethral opening (e.g., at or near the pubic bone). In some embodiments, the opening 106 may exhibit an elongated shape such that about one-third of the opening 106 is above the urethra and about two-thirds of the opening is below the opening 106 during use. The opening 106 may exhibit an elongated shape since the space between the legs of a female is relatively small, thereby only permitting the flow of the fluids along a path that corresponds to the elongated shape of the opening 106. For example, the opening may extend longitudinally along the fluid impermeable barrier. The opening 106 in the fluid impermeable barrier 102 may exhibit a width that is measured transverse to the longitudinal direction and may be at least about 10% of the circumference of the fluid collection device 100, such as about 25% to about 50%, about 40% to about 60%, about 50% to about 75%, about 65% to about 85%, or about 75% to about 100% of the circumference of the fluid collection device 100. The opening 106 may exhibit a width that is greater than 50% of the circumference of the fluid collection device 100 since the vacuum (e.g., suction) through the conduit 108 pulls the fluid into the conduit 108. In some embodiments, the opening 106 may be vertically oriented (e.g., having a major axis parallel to the longitudinal axis of the device 100). In some embodiments, (not shown), the opening 106 may be horizontally oriented (e.g., having a major axis perpendicular to the longitudinal axis of the device 100). In some embodiments, the inward border or edge 129 of the fluid impermeable barrier 102 defines the opening 106. The edge 129 may include two opposing arced portions, the arc portions following the outer circumference or periphery of the substantially cylindrical fluid impermeable barrier 102. In an embodiment, the fluid impermeable barrier 102 may be configured to be attached to the individual, such as adhesively attached (e.g., with a hydrogel adhesive) to the individual.

The fluid impermeable barrier 102 may also temporarily store the fluids in the chamber 104. For example, the fluid impermeable barrier 102 may be formed of any suitable fluid impermeable materials, such as a fluid impermeable polymer (e.g., silicone, polypropylene, polyethylene, polyethylene terephthalate, a polycarbonate, etc.), polyurethane films, thermoplastic elastomer (TPE), rubber, thermoplastic polyurethane, another suitable material, or combinations thereof. As such, the fluid impermeable barrier 102 substantially prevents the fluids from exiting the portions of the chamber 104 that are spaced from the opening 106. The fluid impermeable barrier 102 is flexible, thereby enabling the fluid collection device 100 to bend or curve when positioned against the body of a wearer. Example fluid impermeable barriers may include, but are not limited to, a fluid impermeable barrier including at least one of Versaflex CL 2000X TPE, Dynaflex G6713 TPE, or Silpuran 6000/05 A/B silicone.

In an embodiment, the fluid impermeable barrier 102 may be air permeable. In such an embodiment, the fluid impermeable barrier 102 may be formed of a hydrophobic material that defines a plurality of pores. In an embodiment, one or more portions of at least the outer surface of the fluid impermeable barrier 102 may be formed from a soft and/or smooth material, thereby reducing chaffing. The fluid impermeable barrier 102 may include markings thereon, such as one or more markings to aid a user in aligning the device 100 on the wearer. For example, a line on the fluid impermeable barrier 102 (e.g., opposite the opening 106) may allow a healthcare professional to align the opening 106 over the urethra of the wearer. In examples, the markings may include one or more of alignment guide or an orientation indicator, such as a stripe or hashes. Such markings may be positioned to align the device 100 to one or more anatomical features such as a pubic bone, etc.

The fluid collection device 100 may include a fluid permeable body 120 or layer disposed in the chamber 104. The fluid permeable body 120 may cover or extend across at least a portion (e.g., all) of the opening 106. The fluid permeable body 120 may be configured to wick or otherwise draw any fluid away from the opening 106, thereby preventing the fluid from escaping the chamber 104. The fluid permeable body 120 also may wick the fluid generally towards an interior of the chamber 104, as discussed in more detail below. A portion of the fluid permeable body 120 may define a portion of an outer surface of the fluid collection device 100. Specifically, the portion of the fluid permeable body 120 defining the portion of the outer surface of the fluid collection device 100 may be the portion of the fluid permeable body 120 exposed by the opening 106 defined by the fluid impermeable barrier 102 that contacts the user. Moreover, the portion of the fluid permeable device defining the portion of the outer surface of the fluid collection device 100 may be free from coverage by gauze or other wicking material at the opening.

The fluid permeable body 120 can be configured to wick and/or allow transport of fluid away from the opening 106 towards a reservoir 122 and/or an inlet 110 of the conduit 108. The fluid permeable body 120 may include any material that may wick the fluid. The permeable properties referred to herein may be wicking, capillary action, diffusion, or other similar properties or processes, and are referred to herein as “permeable” and/or “wicking.” Such “wicking” or other physical properties may exclude absorption into the fluid permeable body 120, such as not include absorption of the bodily fluid into the fluid permeable body 120. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the fluid permeable body 120 (e.g., absorbency), such as less than about 30 wt % of the dry weight of the fluid permeable body 110, less than about 20 wt %, less than about 10 wt %, less than about 7 wt %, less than about 5 wt %, less than about 3 wt %, less than about 2 wt %, less than about 1 wt %, or less than about 0.5 wt % of the dry weight of the fluid permeable body 210. In an embodiment, the fluid permeable body 210 may include at least one absorbent or adsorbent material.

The fluid permeable body 120 may include a one-way fluid movement fabric. As such, the fluid permeable body 120 may remove fluid from the area around the female urethra, thereby leaving the urethra dry. The fluid permeable body 120 may enable the fluid to flow generally towards a reservoir 122 (shown in FIG. 1D) in the chamber 104 and/or the inlet 110 of the conduit 108. For example, the fluid permeable body 120 may include a porous or fibrous material, such as hydrophilic polyolefin. In some embodiments, the fluid permeable body 120 consists of or consists essentially of a porous or fibrous material, such as hydrophilic polyolefin. Examples of polyolefin that may be used in the fluid permeable body 120 include, but are not limited to, polyethylene, polypropylene, polyisobutylene, ethylene propylene rubber, ethylene propylene diene monomer, or combinations thereof. The porous or fibrous material may be extruded into a substantially cylindrically shape to fit within the chamber 104 of the fluid impermeable barrier 102. The fluid permeable body 120 may include varying densities or dimensions. Moreover, the fluid permeable body 120 may be manufactured according to various manufacturing methods, such as molding, extrusion, or sintering.

In some embodiments, the fluid permeable body 120 includes a singular and porous body. That is, during use, the fluid permeable body 120 extends from the conduit 108 to interface the fluid impermeable barrier 102 and the opening 106. In some embodiments, a majority of the outer surface 109 (shown in FIG. 1C) of the fluid permeable body 120 interfaces with an inner surface 103 (shown in FIG. 1C) of the fluid impermeable barrier 106. In some embodiments, at least a portion of the singular porous material of the fluid permeable body 120 extends continuously between the opening 106 and the reservoir 122 to wick any fluid from the opening 106 directly to the reservoir 122. Moreover, as the fluid impermeable barrier 102 is flexible and the fluid permeable body 120 is configured to wick fluid from the body rather than absorb fluid from the body and hold the fluid against the body, the fluid collection device 100, in some embodiments, is free from a seal or cushioning ring on the inward edge 129 defining the opening 106. In these and other embodiments, the fluid permeable body 120 includes an outer surface and a single layer or type of material between the opening 106 and the conduit 108 positioned within the fluid permeable body 120.

In some embodiments, the fluid permeable body 120 may include two or more layers of fluid permeable materials and include no (or an absence of) more than two layers of material between the opening 106 and the conduit 108 positioned within the fluid permeable body 120. For example, the fluid collection device 100 may include a fluid permeable membrane covering or wrapping around at least a portion of a fluid permeable body, with both the fluid permeable membrane and the fluid permeable body being disposed in the chamber 104. The fluid permeable membrane may cover or extend across at least a portion (e.g., all) of the opening 106. The fluid permeable membrane may be configured to wick any fluid away from the opening 106, thereby preventing the fluid from escaping the chamber 104. In some embodiments, at least one of the fluid permeable membrane or the fluid permeable support include nylon configured to wick fluid away from the opening 106. The material of the fluid permeable membrane and the fluid permeable support also may include natural fibers. In such examples, the material may have a coating to prevent or limit absorption of fluid into the material, such as a water repellent coating.

The fluid permeable membrane may also wick the fluid generally towards an interior of the chamber 104. The fluid permeable membrane may include any material that may wick the fluid. For example, the fluid permeable membrane may include fabric, such as a gauze (e.g., a silk, linen, polymer based materials such as polyester, or cotton gauze), another soft fabric (e.g., jersey knit fabric or the like), or another smooth fabric (e.g., rayon, satin, or the like). Forming the fluid permeable membrane from gauze, soft fabric, and/or smooth fabric may reduce chaffing caused by the fluid collection device 100. Other embodiments of fluid permeable membranes, fluid permeable supports, chambers, and their shapes and configurations are disclosed in U.S. patent application Ser. No. 15/612,325 filed on Jun. 2, 2017; U.S. patent application Ser. No. 15/260,103 filed on Sep. 8, 2016; U.S. patent application Ser. No. 15/611,587 filed on Jun. 1, 2017; PCT Patent Application No. PCT/US19/29608, filed on Apr. 29, 2019, the disclosure of each of which is incorporated herein, in its entirety, by this reference. In many embodiments, the fluid permeable body 120 includes a fluid permeable support including a porous spun nylon fiber structure and a fluid permeable wicking membrane including gauze at least partially enclosing the spun nylon fiber structure. For example, the fluid permeable body 120 may include a gauze or other wicking fabric positioned to contact the skin of the user through the opening 106. In some embodiments, the gauze or other wicking fabric is wrapped around a body of spun nylon fibers material and/or covering both sides of a substantially planar spun nylon fibers material. In some embodiments, the gauze or other wicking fabric covers the side of substantially planar spun nylon fibers material that is oriented towards the skin of the user.

FIG. 1B is a front view of a fluid collection device 100 in use on a female user 150. In use, the fluid permeable body 120 of the fluid collection device is positioned at least proximate to a urethra of the user 150. The fluid permeable body 120 is disposed within a chamber 104 (shown in FIG. 1D) of the fluid impermeable barrier 102 of the fluid collection device 100 and is exposed to the urethra of the user 150 through the opening 106 in the fluid collection device 100. The fluid collection device 100 may be secured to the user with any of a number of securement systems disclosed herein. Fluids received in the chamber 104 of the fluid collection device 100 from the urethra may be removed through the conduit 108.

FIG. 1D is a cross-sectional view of the fluid collection device 100 taken along line 1-1 of FIG. 1A. The fluid collection device 100 also includes the conduit 108 that is at least partially disposed in the chamber 104. The conduit 108 (e.g., a tube) includes an inlet 110 at a second end region 127 of the fluid impermeable barrier 102 and an outlet 112 at a first end region 125 of the fluid impermeable barrier 102 positioned downstream from the inlet 110. The conduit 108 provides fluid communication between an interior region of the chamber 104 and a fluid storage container (not shown) or a portable vacuum source (not shown). For example, the conduit 108 may directly or indirectly fluidly couple the interior region of the chamber 104 and/or the reservoir 122 with the fluid storage container or the portable vacuum source.

In the illustrated embodiment, the fluid permeable body 120 defines a bore 202 extending through the fluid permeable body 120 from a first body end 121 of the fluid permeable body 120 to a second body end 123 of the fluid permeable body 120 distal to the first body end 120. In other embodiments, the bore 202 extends only partially into the fluid permeable body from the first body end 121 of the fluid permeable body 120.

In the illustrated embodiment, the conduit 108 is at least partially disposed in the chamber 104 and interfaces at least a portion of the bore 202 of the fluid permeable body 120. For example, the conduit 108 may extend into the fluid impermeable barrier 102 from the first end region 125 (e.g., proximate to the outlet 112) and may extend through the bore 202 to the second end region 127 (e.g., opposite the first end region 125) to a point proximate to the reservoir 122 such that the inlet 110 is in fluid communication with the reservoir 122. For example, in the illustrated embodiment, the inlet 110 extends past the second body end 123 and is positioned in the reservoir 122. However, in other embodiments, the inlet 110 may be positioned flush with or behind the second body end 123 of the fluid permeable body 120 that partially defines the reservoir 122. In some embodiments, the second body end 123 extends to the second end region 127 to substantially fill the chamber 104 and cover the inlet 110. The fluid collected in the fluid collection device 100 may be removed from the interior region of the chamber 104 via the conduit 108. The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex.

The fluid impermeable barrier 102 may store fluids in the reservoir 122 therein. The reservoir 122 may be an unoccupied portion of the chamber 104 and is void of other material. In some embodiments, the reservoir 122 is defined at least partially by the fluid permeable body 120 and the fluid impermeable barrier 102. For example, in an embodiment, the reservoir 122 may be located at the portion of the chamber 104 that is closest to the inlet 110 (e.g., the second end region). Accordingly, in the embodiment in FIG. 1D, the reservoir 122 is defined by the second body end 123 of the fluid permeable body 120 and the second end region 127 of the fluid impermeable barrier 122. However, the reservoir 122 may be located at different locations in the chamber 104. For example, the reservoir 122 may be located at the end of the chamber 104 that is closest to the outlet 112. In these and other embodiments, the conduit 108 may extend through the first end region 125 of the fluid impermeable barrier 102 and to the reservoir 122 without extending through the fluid permeable body 120. Accordingly, in these and other embodiments, the fluid permeable body 120 may be free from the bore. In another embodiment, the fluid collection device 100 may include multiple reservoirs, such as a first reservoir that is located at the portion of the chamber of the chamber 104 that is closest to the inlet 110 (e.g., second end region) and a second reservoir that is located at the portion of the of the chamber 104 that is closest to the outlet 112 (e.g., first end region). In another example, the fluid permeable body 120 is spaced from at least a portion of the conduit 108 and the reservoir 122 may be the space between the fluid permeable body 120 and the conduit 108. In some embodiments, the fluid permeable body 120 fills or occupies substantially all of the chamber 104, including filling or occupying substantially all of the reservoir 122 is between the inlet 110 and the second end region 127 of the fluid impermeable barrier 102. Other embodiments of reservoirs, fluid impermeable barriers, fluid permeable membranes, fluid permeable bodies, chambers, and their shapes and configurations are disclosed in U.S. patent application Ser. No. 15/612,325 filed on Jun. 2, 2017; U.S. patent application Ser. No. 15/260,103 filed on Sep. 8, 2016; and U.S. patent application Ser. No. 15/611,587 filed on Jun. 1, 2017, the disclosure of each of which is incorporated herein, in its entirety, by this reference.

The fluid impermeable barrier 102 and the fluid permeable body 120 may be configured to have the conduit 108 at least partially disposed in the chamber 104. For example, the fluid permeable body 120 may be configured to form a space that accommodates the conduit 108, such as the bore 202. In another example, the fluid impermeable barrier 102 may define an aperture 124 sized to receive the conduit 108 (e.g., at least one tube). The at least one conduit 108 may be disposed in the chamber 104 via the aperture 124. The aperture 124 may be configured to form an at least substantially fluid tight seal against the conduit 108 or the at least one tube thereby substantially preventing the fluids from escaping the chamber 104.

In some embodiments, the conduit 108 may extend through the fluid permeable body 120 and at least partially into the reservoir 122, as shown in FIG. 1D. In some embodiments, the conduit 108 may extend through the fluid permeable body 120 and terminate at or before the second body end 123 of the fluid permeable body 120 such that the conduit 108 does not extend into the reservoir 122 (or the reservoir 122 is absent of the conduit 108). For example, an end (e.g., the inlet 110) of the conduit 108 may be generally flush or coplanar with the second body end 123 of the fluid permeable body 120. In other embodiments, the end of the conduit 108 may be recessed from the second body end 123 of the fluid permeable body 120. The end (e.g., the inlet 110) of the conduit 108 also may be selectively moveable between partially extending into the reservoir 122 (shown in FIG. 1D) and recessed from or flush with the second body end 123 of the fluid permeable body.

When secured to the fluid collection device 100, the conduit 108 is configured to provide fluid communication with and at least partially extend between one or more of a fluid storage containers and a portable vacuum source, as described below with FIG. 8. For example, the conduit 108 may be configured to be fluidly coupled to and at least partially extend between one or more of the fluid storage containers and the portable vacuum source. In an embodiment, the conduit 108 is configured to be directly connected to the portable vacuum source. In such an example, the conduit 108 may extend from the fluid impermeable barrier 102 by at least one foot, at least two feet, at least three feet, or at least six feet. In another example, the conduit 108 is configured to be indirectly connected to at least one of the fluid storage container or the portable vacuum source. In some examples, the conduit may be frosted or opaque (e.g., black) to obscure visibility of the fluids therein. In some embodiments, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

The inlet 110 and the outlet 112 are configured to provide fluid communication (e.g., directly or indirectly) between the portable vacuum source (not shown) and the chamber 104 (e.g., the reservoir 122). For example, the inlet 110 and the outlet 112 of the conduit 108 may be configured to directly or indirectly fluidly couple the portable vacuum source to the reservoir 122. In an embodiment, the inlet 110 and/or the outlet 112 may form a male connector. In another example, the inlet 110 and/or the outlet 112 may form a female connector. In an embodiment, the inlet 110 and/or the outlet 112 may include ribs that are configured to facilitate secure couplings. In an embodiment, the inlet 110 and/or the outlet 112 may form a tapered shape. In an embodiment, the inlet 110 and/or the outlet 112 may include a rigid or flexible material.

Locating the inlet 110 at or near a gravimetrically low point of the chamber 104 enables the conduit to receive more of the fluids than if inlet 110 was located elsewhere and reduce the likelihood of pooling (e.g., pooling of the fluids may cause microbe growth and foul odors). For instance, the fluids in the fluid permeable body 120 may flow in any direction due to capillary forces. However, the fluids may exhibit a preference to flow in the direction of gravity, especially when at least a portion of the fluid permeable body 120 is saturated with the fluids.

As the portable vacuum source applies a vacuum/suction in the conduit 108, the fluid(s) in the chamber 104 (e.g., such as in the reservoir 122 positioned at the first end region 125, the second end region 127, or other intermediary positions within the chamber 104) may be drawn into the inlet 110 and out of the fluid collection device 100 via the conduit 108.

In an embodiment, the conduit 108 is configured to be at least insertable into the chamber 104. In such an embodiment, the conduit 108 may include one or more markers 131 (shown in FIG. 1A) on an exterior thereof that are configured to facilitate insertion of the conduit 108 into the chamber 104. For example, the conduit 108 may include one or more markings thereon that are configured to prevent over or under insertion of the conduit 108, such as when the conduit 108 defines an inlet 110 that is configured to be disposed in or adjacent to the reservoir 122. In another embodiment, the conduit 108 may include one or more markings thereon that are configured to facilitate correct rotation of the conduit 108 relative to the chamber 104. In an embodiment, the one or more markings may include a line, a dot, a sticker, or any other suitable marking. In examples, the conduit 108 may extend into the fluid impermeable barrier 102 from the first end region (e.g., proximate to the outlet 112) and may extend to the second end region (e.g., opposite the first end region) to a point proximate to the reservoir 122 such that the inlet 110 is in fluid communication with the reservoir 122. In some embodiments (not shown), the conduit 108 may enter the second end region and the inlet 110 may be disposed in the second end region (e.g., in the reservoir 122). The fluid collected in the fluid collection device 100 may be removed from the interior region of the chamber 104 via the conduit 108. The conduit 108 may include a flexible material such as plastic tubing (e.g., medical tubing) as disclosed herein. In some examples, the conduit 108 may include one or more portions that are resilient, such as having one or more of a diameter or wall thickness that allows the conduit to be flexible.

In an embodiment, one or more components of the fluid collection device 100 may include an antimicrobial material, such as an antibacterial material where the fluid collection device may contact the wearer or the bodily fluid of the wearer. The antimicrobial material may include an antimicrobial coating, such as a nitrofurazone or silver coating. The antimicrobial material may inhibit microbial growth, such as microbial growth due to pooling or stagnation of the fluids. In an embodiment, one or more components of the fluid collection device 100 (e. g., impermeable barrier 102, conduit 108, etc.) may include an odor blocking or absorbing material such as a cyclodextrine containing material or a thermoplastic elastomer (TPE) polymer.

In any of the embodiments disclosed herein, the conduits 108 may include or be operably coupled to a flow meter (not shown) to measure the flow of fluids therein, one or more securement devices (e.g., a StatLock securement device, not shown) or fittings to secure the conduit 108 to one or more components of the systems or devices disclosed herein (e.g., portable vacuum source or fluid storage container), or one or more valves to control the flow of fluids in the systems and devices herein. In an embodiment, at least one of portion of the conduit 108 of the fluid collection devices or systems herein may be formed of an at least partially opaque material which may obscure the fluids that are present therein. For example, a first section of the conduit 108 disclosed herein may be formed of an opaque material or translucent material while a second section of the conduit 108 may be formed of a transparent material or translucent material. In some embodiments, the first section may include transparent or translucent material. Unlike the opaque or nearly opaque material, the translucent material allows a user of the devices and systems herein to visually identify fluids or issues that are inhibiting the flow of fluids within the conduit 108.

In any of the examples, systems or devices disclosed herein, the system of fluid collection device may include moisture sensors (not shown) disposed inside of the chamber of the fluid collection device. In such examples, the moisture sensor may be operably coupled to a controller or directly to the portable vacuum source, and may provide electrical signals indicating that moisture is or is not detected in one or more portions of the chamber. The moisture sensor(s) may provide an indication that moisture is present, and responsive thereto, the controller or portable vacuum device may direct the initiation of suction to the chamber to remove the fluid therefrom. Suitable moisture sensors may include capacitance sensors, volumetric sensors, potential sensors, resistance sensors, frequency domain reflectometry sensors, time domain reflectometry sensors, or any other suitable moisture sensor. In practice, the moisture sensors may detect moisture in the chamber and may provide a signal to the controller or portable vacuum source to activate the portable suction device.

FIG. 1E is a block diagram of a system 10 for fluid collection, according to an embodiment. The system 10 may utilized with any of the holders disclosed herein. The system 10 includes a fluid collection device 12, a fluid storage container 14, and a portable vacuum source 16. The fluid collection device 12 may include any of the fluid collection devices disclosed herein, such as the fluid collection device 100. The fluid collection device 12 in the system 10 may be secured to any of the holders disclosed herein. The fluid collection device 12, the fluid storage container 14, and the portable vacuum source 16 may be fluidly coupled to each other via one or more conduits 17. The conduit 17 may include any of the conduits disclosed herein, such as the conduit 108. The fluid collection device 12 may be operably coupled to one or more of the fluid storage container 14 or the portable vacuum source via the conduit 17. Fluid (e.g., urine or other bodily fluids) collected in the fluid collection device 12 may be removed from the fluid collection device 12 via the conduit 17, which protrudes into an interior region of the fluid collection device 12. For example, a first open end of the conduit 17 may extend into the fluid collection device 12 to a reservoir therein. The second open end of the conduit 17 may extend into the fluid storage container 14 or the portable vacuum source 16. The suction force may be introduced into the interior region of the fluid collection device 12 via the first open end of the conduit 17 responsive to a suction (e.g., vacuum) force applied at the second end of the conduit 17. The suction force may be applied to the second open end of the conduit 17 by the portable vacuum source 16 either directly or indirectly.

The suction force may be applied indirectly via the fluid storage container 14. For example, the second open end of the conduit 17 may be disposed within the fluid storage container 14 and an additional conduit 17 may extend from the fluid storage container 14 to the portable vacuum source 16. Accordingly, the portable vacuum source 16 may apply suction to the fluid collection device 12 via the fluid storage container 14. The suction force may be applied directly via the fluid storage container 14. For example, the second open end of the conduit 17 may be disposed within the portable vacuum source 16. An additional conduit 17 may extend from the portable vacuum source 16 to a point outside of the fluid collection device 12, such as to the fluid storage container 14. In such examples, the portable vacuum source 16 may be disposed between the fluid collection device 12 and the fluid storage container 14.

The fluid collection device 12 may be shaped and sized to be positioned adjacent or proximate to a female urethra. Any of the holders disclosed herein may be used to hold or retain the fluid collection device adjacent or proximate to the female urethra. The fluid collection member of the fluid collection device 12 may include a fluid impermeable barrier at least partially defining a chamber (e.g., interior region of the fluid collection device member) of the fluid collection device 12. As described in more detail above, the fluid collection device 12 may include a softer, thinner fluid impermeable barrier than conventional fluid collection devices. The fluid impermeable barrier also defines an opening extending therethrough from the external environment. The opening may be positioned on the fluid collection member to be aligned adjacent or proximate to a female urethra. The fluid collection member of the fluid collection device 12 may include a fluid permeable body disposed within the fluid impermeable barrier. The fluid permeably body may include a fluid permeable membrane and fluid permeable support disposed within the fluid permeable membrane. The conduit 17 may extend into the fluid collection device 12 at a first end region, through one or more of the fluid impermeable barrier, fluid permeable membrane, or the fluid permeable support to a second end region of the fluid collection member of the fluid collection device 12. Example fluid collection devices for use with the systems and methods herein are described in more detail below.

In some embodiments, the fluid storage container 14 may include a bag (e. g., drainage bag), a bottle or cup (e.g., collection jar), or any other enclosed container for storing bodily fluids such as urine. In examples, the conduit 17 may extend from the fluid collection device 12 and attach to the fluid storage container 14 at a first point therein. An additional conduit 17 may attach to the fluid storage container 14 at a second point thereon and may extend and attach to the portable vacuum source 16. For example, the fluid storage container 14 may include a container fluidly coupled to a first conduit section that is also fluidly coupled to the fluid collection member of the fluid collection device 12. The container may be fluidly coupled to a second section of the conduit 17 that is also fluidly coupled to a portable vacuum source. In such examples, the portable vacuum source 16 may provide a vacuum/suction through the container to the fluid collection member to provide suction in the chamber of the fluid collection member. Accordingly, a vacuum (e.g., suction) may be drawn through fluid collection device 12 via the fluid storage container 14. As the fluid is drained from the chamber, the fluid may travel through the first section of conduit to the fluid storage container where it may be retained. Fluid, such as urine, may be drained from the fluid collection device 12 using the portable vacuum source 16.

In some embodiments, the portable vacuum source 16 may be disposed in or on the fluid collection device 12. In such examples, the conduit 17 may extend from the fluid collection device and attach to the portable vacuum source 16 at a first point therein. An additional conduit 17 may attach to the portable vacuum source 16 at a second point thereon and may extend out of the fluid collection device 12, and may attach to the fluid storage container 14. Accordingly, a vacuum (e.g., suction) may be drawn through fluid collection device 12 via the fluid storage container 14.

The portable vacuum source 16 may include one or more of a manual vacuum pump, and electric vacuum pump, a diaphragm pump, a centrifugal pump, a displacement pump, a magnetically driven pump, a peristaltic pump, or any pump configured to produce a vacuum. The portable vacuum source 16 may provide a vacuum or suction to remove fluid from the fluid collection member of the fluid collection device 12. In some embodiments, the portable vacuum source 16 may be powered by one or more of a power cord (e.g., connected to a power socket), one or more batteries, or even manual power (e.g., a hand operated vacuum pump). In examples, the portable vacuum source 16 may be sized and shaped to fit outside of, on, or within the fluid collection device 12. For example, the portable vacuum source 16 may include one or more miniaturized pumps or one or more micro pumps. The portable vacuum sources 16 disclosed herein may include one or more of a switch, a button, a plug, a remote, or any other device suitable to activate the portable vacuum source 16. It should be understood that the portable vacuum sources 16 disclosed herein may provide a portable means of providing a suction or vacuum that allows use of the devices and systems herein outside of hospital or care facility environments where vacuum lines are plumbed into patient rooms or large (e.g., larger or heavier than a patient can readily carry) vacuum sources are located. For example, a portable vacuum source may be small and light enough to be carried by a user (e.g., patient) or aid (e.g., nurse) during transportation of the user.

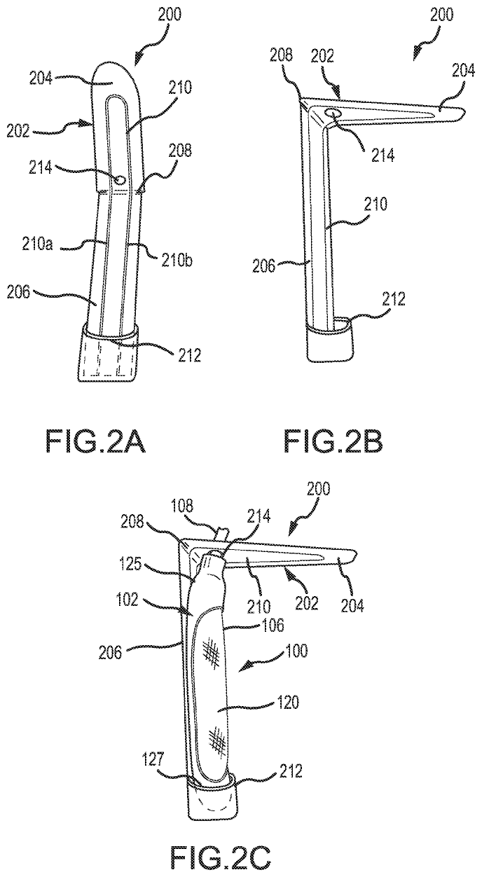

A holder may be used to hold the fluid collection device 12, 100 in a position to effectively receive fluid discharged from a user. FIGS. 2A-2C are various views of a holder 200 that assists in maintaining the correct and most effective position of a fluid collection device during use. Specifically, FIG. 2A is a front view of a holder in a partially bent position, FIG. 2B is an isometric side view of the holder of FIG. 2A in a bent position, and FIG. 2C is an isometric side view of a fluid collection assembly including the holder of FIG. 2A in a bent position with the fluid collection device 100 secured thereto, according to an embodiment. While the fluid collection assembly includes the fluid collection device 100 secured to the holder 200, fluid collection assemblies of this disclosure may include any one of various fluid collection devices known in the art secured to the holder 200.

In many embodiments, the holder 200 is configured to selectively and detachably secure to the fluid collection device 100. The holder 200 may include a holder body 202 a shape memory material 210, and a securement element. The holder body 202 is elongated and includes a first (e.g. proximal) region 204 and a second (e.g., distal) region 206, according to an embodiment. The first region 204 is configured to be disposed at least proximate to a supra-pubic region of the user, according to an embodiment. The second region 206 may be adjustable relative to the first region 204, and configured to hold the opening 106 of the fluid collection device 100 at least proximate to the urethra of the user when the first region 204 is disposed at least proximate to the supra-pubic region of the user. In many embodiments, the holder body 202 is adjustable between at least a generally planar configuration and a substantially L-shaped configuration (shown in FIG. 2B). In the L-shaped configuration, a fold 208 or crease separates the first region 204 from the second region 206.

In many embodiments, the holder body 202 includes a fluid impermeable material. For example, the holder body 202 may include any of the materials described above in reference to the fluid impermeable barrier 102 of the fluid collection device 100. In some embodiments, material of the holder body 202 includes one or more of a flexible or pliable plastic configured to bend without breaking, a silicone material, a paper material, a fabric material, or combinations thereof. The material or materials of the holder body 202 may be configured to resist absorption, and may instead be waterproof or include a waterproof coating on any of the described materials. The holder body 202 may be flexible or bendable about the fold 208 or crease. In some embodiments, the fold 208 or crease is configured to promote or allow pivoting or folding of holder body 202 about the fold 208. For example, the fold 208 may include a flexible region or a hinge. In some embodiments, the holder body 202 is generally uniformly flexible along the longitudinal length of the shape memory material 210 such that the holder body may conform to the contours of the user.

The holder 202 may include an elongated shape. For example, the holder 202 may include an elongated shape sufficient that during use of the holder 200, the first region 204 extends from approximately the pubic bone of the user at least partially towards the navel of the user, while the second region 206 extends from the first region at a length sufficient to hold at least some (e.g., substantially all) of the fluid impermeable barrier 102 or shell/casing of the fluid collection device between the first region 204 of the holder 202 and a distal end of the second region 206. In some embodiments, the holder 202 includes a longitudinal length of about 20 cm to about 40 cm, about 20 cm to about 30 cm, about 25 cm to about 35 cm, about 30 cm to about 40 cm, about 22 cm to about 27 cm, about 27 cm to about 32 cm, about 32 cm to about 37 cm, about 25 cm to about 27 cm, about 27 to about 29 cm, about 29 cm to about 31 cm, about 31 cm to about 33 cm, or about 33 to about 35 cm.

As noted above, the first region 204 extends from approximately the pubic bone of the user at least partially towards the navel of the user. For example, the first region 204 may include a longitudinal length (from a terminating proximal end of the first region 204 to the fold 208) of about 10 cm to about 20 cm, about 10 cm to about 15 cm, about 12.5 cm to about 17.5 cm, about 15 cm to about 20 cm, about 12 cm to about 14 cm, about 14 cm to about 16 cm, about 16 cm to about 18 cm, about 12 cm to about 13 cm, about 13 cm to about 14 cm, about 14 cm to about 15 cm, about 15 cm to about 16 cm, about 16 cm to about 17 cm, or about 17 cm to about 18 cm.

As noted above, the second region 206 extends from the first region at a length sufficient to hold at least some (e.g., substantially all) of the fluid impermeable barrier 102 or shell/casing of the fluid collection device between the first region 204 of the holder 202 and a distal end of the second region 206. Accordingly, the second region 206 may exhibit a longitudinal length that extend from a distal location below the urethral opening (e.g., at or near the anus or the vaginal opening) to a second location above the urethral opening (e.g., at or near the clitoris, the pubic hair, or the pubic bone). In some embodiments, the second region 206 may include a longitudinal length (from a terminating distal end of the second region 206 to the fold 208) of about 12 cm to about 12 cm, about 12 cm to about 17 cm, about 14.5 cm to about 16.5 cm, about 17 cm to about 22 cm, about 14 cm to about 16 cm, about 16 cm to about 18 cm, about 18 cm to about 20 cm, about 14 cm to about 15 cm, about 15 cm to about 16 cm, about 16 cm to about 17 cm, about 17 cm to about 18 cm, about 18 cm to about 19 cm, or about 19 cm to about 20 cm.

In some embodiments, the holder body 202 includes a lateral width of about 1 cm to about 5 cm, about 1 cm to about 3 cm, about 2 cm to about 4 cm, about 3 cm to about 5 cm, about 1.5 cm to about 2 cm, about 2 cm to about 2.5 cm, about 2.5 cm to about 3 cm, about 3 cm to about 3.5 cm, or about 3.5 cm to about 4 cm. The lateral width of the holder body 202 may be uniform throughout the holder body 202 or may taper at one or both ends of the holder body 202.

The holder 200 also includes a shape memory material 210 such as a shape memory polymer or a metal (e.g., shape memory metal), according to an embodiment. The shape memory material 210 may extend longitudinally along the holder body at least partially in the first region 204 and at least partially in the second region 206. In many embodiments, the shape memory material 210 is configured to bend and/or contour the holder body 202 to a substantially L-shaped configuration effective to dispose the first region 204 of the holder body 202 at least proximate to the supra-pubic region of the user while the second region 206 of the holder body 202 holds the opening 106 of the fluid collection device 100 at least proximate to the urethra of the user. The shape memory material 210 may be configured to bend, contour, or conform the holder body 202 to the unique shape of the body of the user.

Suitable shape memory materials are composed to adopt an intermediate or permanent shape in response to a stimuli. The stimuli may include an external physical force (e.g., bending force), heat, electrical bias, or a magnetic field. While the term “shape memory” is used to describe some of the “shape memory materials” herein, it should be understood that, in some examples, the material modified by the term “shape memory” may not necessarily need to return to a preselected shape upon application of a stimuli, as understood as the classical definition of the “shape memory material.” Rather, at least some of the shape memory materials herein may simply hold a selected shape when bent, set, or cured into a specific shape and/or when cooled in a specific shape, regardless of the stimuli applied thereto after. The shape memory materials may be returned to the original shape or changed to a new shape by application of stimuli. For example, a metal wire bent to a first shape may be utilized as the shape memory material, whereinafter the metal wire may be modified to a second shape via physical force applied thereto or via heating.

In an embodiment, the shape memory material may include metal, such as an elemental metal, an alloy, or shape memory alloy. Suitable shape memory metals may include standard steels, stainless steel, carbon alloy steel, head treated steel, aluminum, silver, copper, iron, nickel, zinc, tin, beryllium, or the like. Suitable shape memory alloys may include stainless steel; galvanized steel; aluminum alloys; nickel-titanium alloys, such as Nitinol, Ni—Ti—Cu, Ni—Ti, Co, or the like; copper-based alloys such as Cu—Zn—Al, Cu—Al—Ni, Cu—Al—Sn, or the like; Co—Cr—Ni—Mo alloys (e.g., Elgiloy®) or the like; or any other alloy having shape memory characteristics. In a particular embodiment, the shape memory material includes 18-20 gauge steel wire. As explained above, the shape memory metals or alloys may merely be metals or alloys that may be shaped to a selected configuration. In some examples, the shape memory metals or alloys may return to a primary shape when an external stimuli is applied thereto. In some examples, the outer surface of the shape memory metal may be coated with a polymer, anodized, passivated, or otherwise treated to prevent corrosion.

Shape memory polymers (“SMPs”) may include polyurethane-based SMPs such as a copolymer (e.g., copolyester, polyurethane, polyetherester, etc.) including blocks of one or more of poly(ε-caprolactone), polyethyleneterephthalate (PET), polyethyleneoxide (PEO), polyethylene glycol (PEG), polystyrene, polymethylmethacrylate (PMMA), polybutylmethacrylate (PBMA), poly(N,N-butadiene), poly(N-methyl-N-oxazoline), polytetrahydrofuran, or poly(butylene terephthalate); thermoplastic polymers such as polyether ether ketone (PEEK), nylon, acetal, polytetrafluoroethylene (PTFE), polysulphone, or the like; polynorbonene; other deformable polymers; or any other shape memory polymer.

In some embodiments, the shape memory material 210 may be at least partially (e.g. entirely) embedded in holder body 202 such that all of the shape memory material 210 covered by at least some of the holder body 202 (e.g., none of the shape memory material 210 is exposed on the holder 200). The shape memory material 210 may be disposed in various shapes and configurations in the holder body 202. In some embodiments, the shape memory material 210 includes at least two longitudinal segments 210a, 210b extending longitudinally along the holder body 202 at least partially in the first region 204 and at least partially in the second region 206. For example, the shape memory material 210 may be formed and/or disposed as an elongated U-shape, oval, or square extending longitudinally along (e.g., in) the holder body 202 at least partially in the first region 204 and at least partially in the second region 206 and including the at least two longitudinal segments 210a, 210b. In some embodiments (not shown), the shape memory material 210 may be formed or disposed as two or more elongated ovals or squares extending longitudinally along the holder body 202 at least partially in the first region 204 and at least partially in the second region 206, and including the at least two longitudinal segments 210a, 210b.

In some embodiments, the shape memory material 210 is positioned along or in most of the longitudinal length of the holder body 202. For example, the shape memory material 210 may be positioned along or in at least 50% of the longitudinal length of the holder body 202, at least 75% of the longitudinal length of the holder body 202, at least 90% of the longitudinal length of the holder body 202, or at least 95% of the longitudinal length of the holder body 202. More particularly, the shape memory material 210 may be positioned along or in at least 50% of the longitudinal length of the first region 204, at least 75% of the longitudinal length of the first region 204, at least 90% of the longitudinal length of the first region 204, or at least 95% of the longitudinal length of the first region 204. Similarly, the shape memory material 210 may be positioned along or in at least 50% of the longitudinal length of the second region 206, at least 75% of the longitudinal length of the second region 206, at least 90% of the longitudinal length of the second region 206, or at least 95% of the longitudinal length of the second region 206.

The holder 200 also may include a slot or a hole 214 disposed at least partially in the first region 204 of the holder body 202 and between two of the at least longitudinal segments 210a, 210b of the shape memory material 210. The slot or the hole 214 may be sized and dimensioned to receive at least the conduit 108 of the fluid collection device 100 therethrough. In the holder 200, the holder body 202 defines a hole 214 (e.g., through hole) extending through the first region 204 between the two elongated segments 210a, 210b and inside the elongated oval the shape memory material 210. In some embodiments, a slot may extend from a terminating proximal end of the first region 204 to the hole 214. For example, in embodiments having the shape memory material 210 disposed as multiple elongated ovals or squares, or two linear segments that do not connect to one another in the first region 204, a slot may extend from a terminating proximal end of the first region 204 to the hole 214. As illustrated in FIG. 2C, the hole 214 (or slot) is positioned and configured such that when the first region 204 is bent or adjusted relative to the second region 206, at least the conduit 108 extends through hole 214 rather than interfering with the first region 204. In some embodiments, the hole 214 is sized substantially complementary to a cross-sectional profile of the conduit 108 such that only the conduit 108 fits through the hole 214. In some embodiments, the hole 214 is sized to allow the conduit 108 and at least some of the proximal end 125 of the fluid impermeable barrier 102 of the fluid collection device 100 to extend through the hole 214.

The holder 200 also may include a securement element configured to secure the second region 206 of the holder body 202 to the fluid collection device 100. For example, the holder 200 includes a pocket 212 on the second region 206 of the holder body 202. The pocket 212 may be sized to hold a distal end 127 of the fluid impermeable barrier 102 of the fluid collection device 100 therein without covering the opening 106 of the fluid collection device 100. The pocket 212 may include any of a number of different materials configured to hold the distal end 127 of the fluid impermeable barrier 102 (or fluid collection device 100) therein. In some embodiments, the pocket 212 includes the same material as the holder body 202. The distal end of the fluid collection device 100 may be friction fit in the pocket 212. In some embodiments, the pocket 212 includes an elastic material such that the pocket stretches to hold the distal end of the fluid collection device 100 therein.

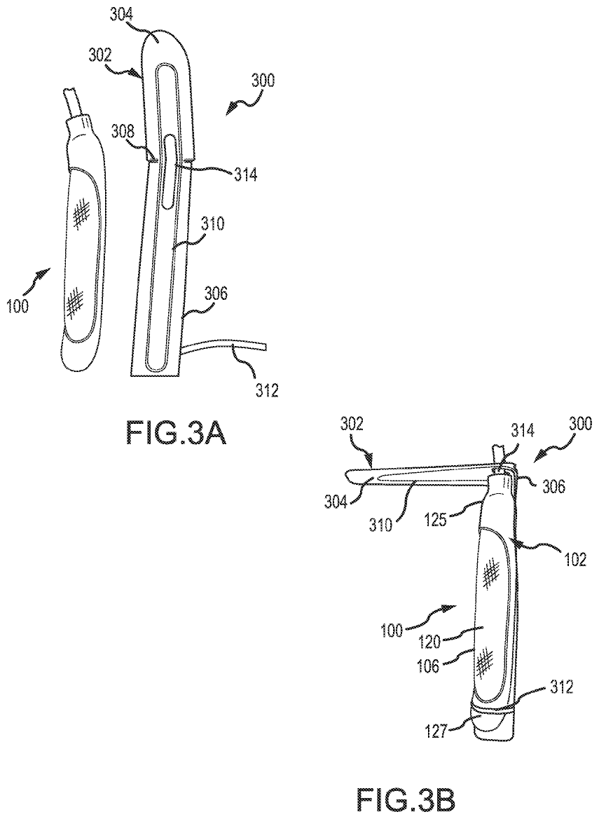

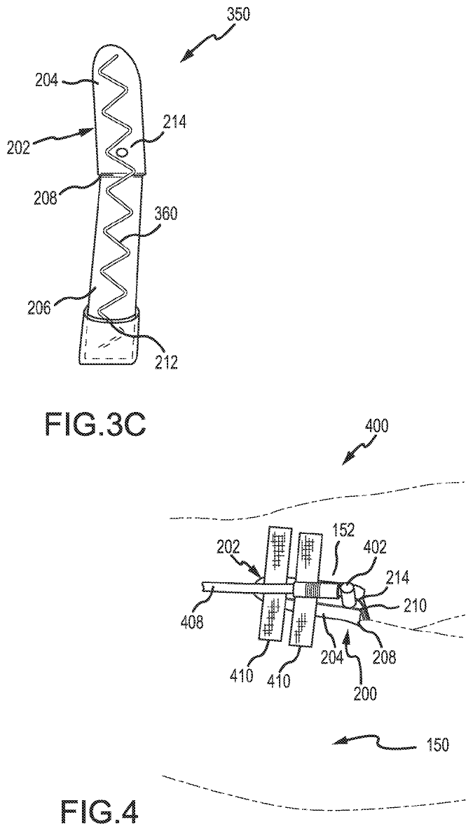

Holders according to this disclosure may include other various configurations of the slot or hole, the shape memory material, and/or the securement element. FIG. 3A is a front view of a holder 300 in a partially bent position and a fluid collection device 100, and FIG. 3B is an isometric side view of a fluid collection assembly including the holder 300 of FIG. 3A in a bent position with the fluid collection device 100 secured thereto, according to an embodiment. Unless otherwise noted, the holder 300 may include any aspect of the holder 200. For example, the holder 300 may include a holder body 302 having a first region 304, a second region 306, and a fold 308 or crease between the first region 304 and the second region 306. The holder body 302, the first region 304, the second region 306, and fold 308 or crease may include any aspect of the holder body 202, the first region 204, the second region 206, and/or the fold 208 or crease of the holder 200. Furthermore, the holder 300 also may include a shape memory material 310. The shape memory material 310 may include any aspect of the shape memory material 210 of the holder 200.

The holder 300 also may include a slot 314 disposed at least partially in the first region 304 of the holder body 302 and between two of the at least longitudinal segments of the shape memory material 310. The slot 314 may be sized and dimensioned to receive at least the conduit 108 of the fluid collection device 100 therethrough. In the holder 300, the holder body 302 defines a slot 314 that extends through a portion of the first region 304 and a portion of the second region 306 between the two elongated segments of the shape memory material 310. For example, the slot 314 may extend through the fold 308 or crease and at least partially into the first region 304 and at least partially into the second region 306. The slot 314 may be positioned and sized such that when the first region 304 is bent or adjusted relative to the second region 306, the conduit 108 and a portion of the proximal end 125 of the fluid collection device 100 may extend through slot 314 rather than interfering with the first region 304. In some embodiments, the slot 314 may extend from a terminating proximal end of the first region 304, past the fold 308, and at least partially into the second region 306. For example, in embodiments having the shape memory material 310 disposed as multiple elongated ovals or squares, or two linear segments that do not connect to one another in the first region 304, the slot 314 may extend from a terminating proximal end of the first region 304, past the fold 308, and at least partially into the second region 306.

The holder 300 also may include a securement element configured to secure the second region 306 of the holder body 302 to the fluid collection device 100. For example, the holder 300 includes a strap 312 on the second region 306 of the holder body 302. The strap 312 may be positioned on the second region 306 of the holder body 302 to extend at least partially around the distal end 127 of the fluid impermeable barrier 102 (or the distal end of the fluid collection device 100 generally) and hold the distal end 127 between the strap 312 and the second region 306 of the holder body 302 without covering the opening 106 of the fluid collection device 100, as illustrated in FIG. 3B. The strap 312 may include any of a number of different materials configured to hold the distal end 127 of the fluid impermeable barrier 102 (or fluid collection device 100) therein. In some embodiments, the strap 312 includes the same material as the holder body 302. The distal end of the fluid collection device 100 may be friction fit in the strap 312. In some embodiments, the strap 312 includes an elastic material such that the strap 312 stretches to hold the distal end of the fluid collection device 100 therein.

FIG. 3C is a front view of a holder 350 in a partially bent position, according to an embodiment. Unless otherwise noted, the holder 350 may include any aspect of the holders 200, 300. For example, the holder 350 may include a holder body 202 having a first region 204, a second region 206, a fold 208 or crease between the first region 204 and the second region 206, a hole 214 extending through the holder body 202, and a pocket 212 secured to the second region 206 of the holder body 202.

Furthermore, the holder 360 also may include a shape memory material 360. Unless otherwise noted, the shape memory material 360 may include any aspect of the shape memory material 210 of the holder 200. In some embodiments, the shape memory material 360 may be disposed in an orientation or configuration that is generally serpentine-like, zig-zag, or sinusoidal wave-like at least partially between the first region 204 and the second region 206. The hole 214 may be disposed at least partially in the first region 204 of the holder body 202 and between adjacent curves of the serpentine orientation or adjacent peaks of the zig-zag orientation of the shape memory material 360.

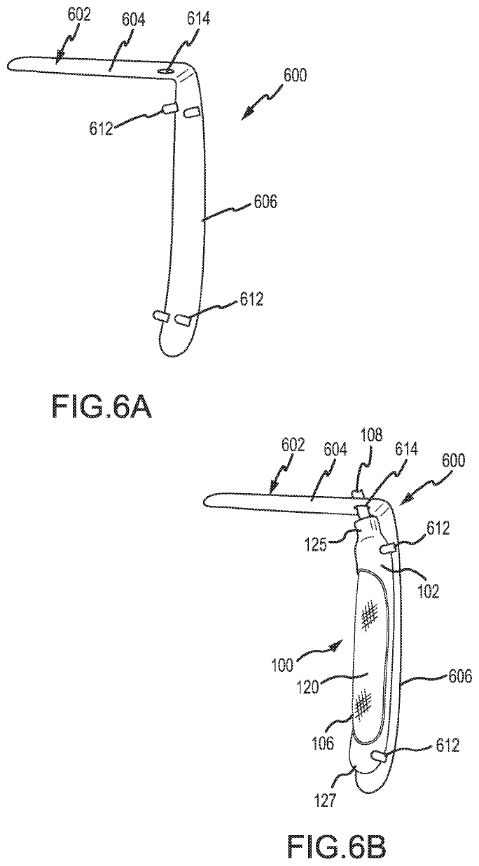

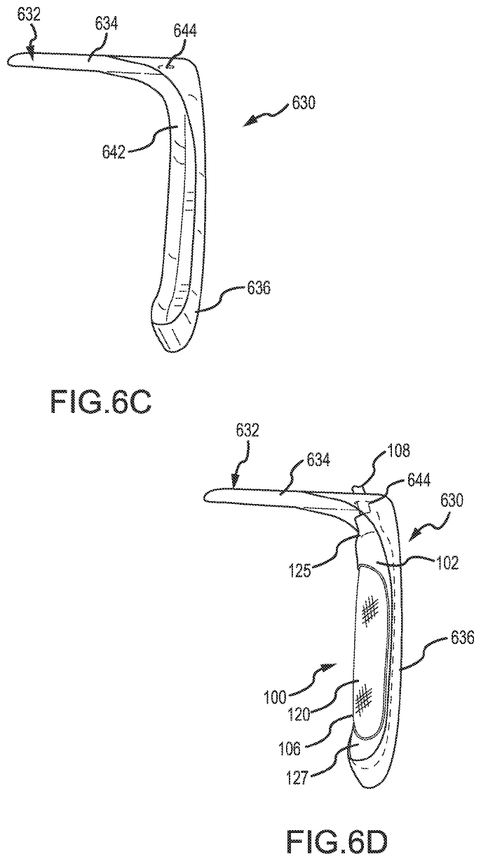

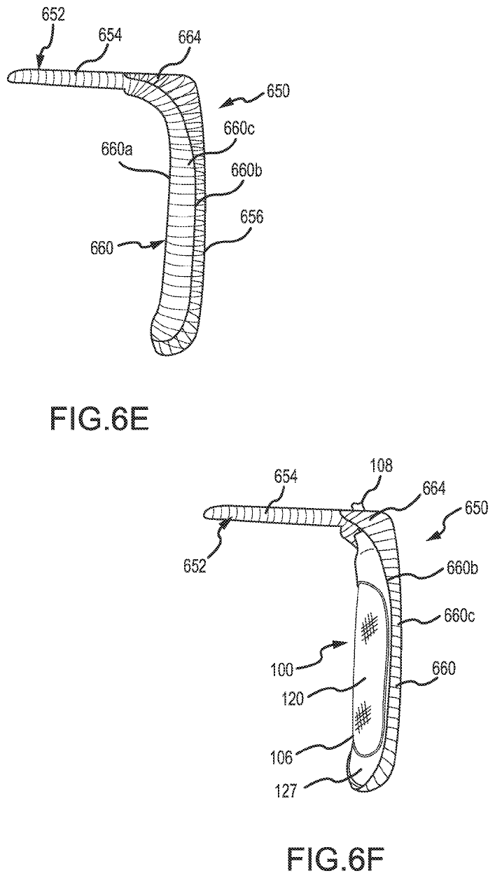

In some embodiments, a holder according to this disclosure may include prongs, molds, and/or different shape memory material configurations. Turning ahead in the drawings, FIG. 6A is an isometric side view of a holder 600 of a fluid collection assembly in a bent position, and FIG. 6B is an isometric side view of a fluid collection device 100 secured to the holder 600 of FIG. 6A, according to an embodiment. Unless otherwise noted, the holder 600 may include any aspect of the holders 200, 300, 350. For example, the holder 600 may include a holder body 602 having a first region 604, a second region 606, a fold or crease between the first region 604 and the second region 606, and a hole 614 in the first region 604. The holder body 602, the first region 604, the second region 606, and fold or crease may include any aspect of the holder body, the first region, the second region, and/or the fold or crease of any other holder disclosed herein. Furthermore, the holder 600 also may include a shape memory material (not visible). The shape memory material of the holder 600 may include any aspect (material, orientations, etc.) of the shape memory material 210 of the holder 200 or the shape memory material of any other holder disclosed herein.

The holder 600 also may include one or more securement elements configured to secure the second region 606 of the holder body 602 to the fluid collection device 100. For example, the holder 600 includes a plurality of prongs 612. Each of the prongs 612 may be adjustable. For example, each of the prongs 612 may include any of the shape memory materials described above. In some embodiments, the holder 600 includes two distal prongs 612 disposed on a distal region of the second region 606 (distal to the first region 605). As shown in FIG. 6B, the two distal prongs 612 may be disposed on the second region 606 of the holder body 602 to have the distal end 127 of the fluid impermeable barrier 102 disposed between the two distal prongs 612. The two distal prongs 612 may be adjusted (e.g., pinched closer together) to hold the distal end 127 between the two distal prongs 612 without covering the opening 106 of the fluid collection device 100. The holder 600 also may include two proximal prongs disposed on a proximal region of the second region 606 (near the first region 604) to have the proximal end 125 of the fluid impermeable barrier 102 disposed between the two proximal prongs 612. The two proximal prongs 612 may be adjusted (e.g., pinched closer together) to hold the proximal end 125 between the two proximal prongs 612 without covering the opening 106 of the fluid collection device 100.

FIG. 6C is an isometric side view of a holder 630 of a fluid collection assembly in a bent position, and FIG. 6D is an isometric side view of a fluid collection device 150 secured to the holder 630 of FIG. 6C, according to an embodiment. Unless otherwise noted, the holder 630 may include any aspect of the holders 200, 300, 350, 600. For example, the holder 630 may include a holder body 632 having a first region 634, a second region 636, a fold or crease between the first region 634 and the second region 636, and a hole 644 in the first region 634. The holder body 632, the first region 634, the second region 636, and fold or crease may include any aspect of the holder body, the first region, the second region, and/or the fold or crease of any other holder disclosed herein. Furthermore, the holder 630 also may include a shape memory material (not visible). The shape memory material of the holder 630 may include any aspect (material, orientations, etc.) of the shape memory material 210 of the holder 200 or the shape memory material of any other holder disclosed herein.

The holder 630 also may include one or more securement elements configured to secure the second region 636 of the holder body 632 to the fluid collection device 100. In some embodiments, the securement element of the holder body 632 may include a recess 642 molded into the second region 636 of the holder body 632. For example, the second region 636 may be molded to include the recess 642 shaped and sized complementary to at least a portion of the fluid collection device 100 such that the fluid collection device 100 may be inserted at least partially into the recess 642 to secure the fluid collection device 100 to the holder 630. In some embodiments, the recess 642 is sized such that, when the fluid collection device 100 is secured to the second region 636, the opening 106 remains uncovered (e.g., the second region 636 of the holder body 632 does not cover the opening 106 of the fluid collection device 100). In some embodiments, the recess 642 is sized such that the edge or perimeter of the second region 636 is almost or substantially flush with the edge or perimeter of the fluid impermeable barrier 102 defining the opening 106 when the fluid collection device 100 is disposed in the recess 642. The recess 642 may include a distal pocket sized and dimensioned to hold at least a portion of the distal end 127 of the fluid impermeable barrier 102 therein without covering the opening 106 of the fluid collection device 100.

FIG. 6E is an isometric side view of a holder 650 of a fluid collection assembly in a bent position, and FIG. 6F is an isometric side view of a fluid collection device 100 secured to the holder 650 of FIG. 6E, according to an embodiment. Unless otherwise noted, the holder 650 may include any aspect of the holders 200, 300, 350, 600, 630. For example, the holder 650 may include a holder body 652 having a first region 654, a second region 656, a fold or crease between the first region 654 and the second region 656, and a hole 664 in the first region 654. The holder body 652, the first region 654, the second region 656, and fold or crease may include any aspect of the holder body, the first region, the second region, and/or the fold or crease of any other holder disclosed herein. Furthermore, the holder 650 also may include a shape memory material 660. The shape memory material 660 of the holder 650 may include any aspect (material) of the shape memory material 210 of the holder 200 or the shape memory material of any other holder disclosed herein.

In some embodiments, the shape memory material 660 may be at least partially (e.g. entirely) embedded in holder body 652 such that all of the shape memory material 660 covered by at least some of the holder body 652 (e.g., none of the shape memory material 660 is exposed on the holder 650). The shape memory material 660 may be disposed in various shapes and configurations in the holder body 652. In some embodiments, the shape memory material 660 includes at least two longitudinal segments 660a, 660b extending longitudinally along the holder body 602 at least partially in the first region 654 and at least partially in the second region 656. In some embodiments, the at least two longitudinal segments 660a, 660b are disposed proximate to an edge or perimeter of the holder body 652. The shape memory material 660 also may include a plurality of lateral segments 660c extending between the two longitudinal segments 660a, 660b.

The shape memory material 660 in the holder body 652 may be configured such that holder body 652 is not only bendable to adjust the first region 654 relative to the second region 656 and/or bendable to conform to the body of the user, but the holder body 652 also may be configured to be bendable to bend at least the second region 656 to conform or contour to the shape of at least a portion of the fluid impermeable barrier 102 of the fluid collection device 100. For example, the lateral segment 660c in the second region 656 of the holder body 652 may bend to contour the second region 656 complementary to at least a portion of the fluid impermeable barrier 102 of the fluid collection device 100 to secured the fluid collection device 100 to the second region 656 of the holder body 652. In some embodiments, the lateral segments 660c in the second region 656 may be bent to form a recess that is shaped and sized complementary to at least a portion of the fluid collection device 100 such that the fluid collection device 100 may be held at least partially in the recess to secure the fluid collection device 100 to the holder 650. In some embodiments, the second region 656 is sized such that, when the lateral segments 660c are bent to secure the fluid collection device 100 to the second region 656, the opening 106 remains uncovered (e.g., the second region 656 of the holder body 652 does not cover the opening 106 of the fluid collection device 100). In some embodiments, the second region 656 is sized such that, when the lateral segments 660c are bent to secure the fluid collection device 100 to the second region 656, the edge or perimeter of the second region 656 is almost or substantially flush with the edge or perimeter of the fluid impermeable barrier 102 defining the opening 106 when the fluid collection device 100 is secured to the second region 656. The second region 656, the longitudinal segments 660a, 660b, and the lateral segment 660c, may be sized such that, when the lateral segments 660c and/or the longitudinal segments are bent to secure the fluid collection device 100 to the second region 656 a distal pocket may be formed to hold at least a portion of the distal end 127 of the fluid impermeable barrier 102 therein without covering the opening 106 of the fluid collection device 100.

FIG. 4 is an isometric view of fluid collection device 100 and the holder 200 of FIG. 2A in use on a user, according to an embodiment. While the fluid collection assembly 400 of FIG. 4 includes the holder 200, other embodiments of holder disclosed herein, such as the holders 300, 350, 610, 630, 650 may be similarly used. The fluid collection assembly 400 is shown secured to the user 150. The first region 204 of the holder body 202 may be configured to be disposed at least proximate to a supra-pubic region 152 of the user 150, and the second region 206 of the holder body 202 may be adjustable relative to the first region 206 and configured to hold the opening 106 (not visible) of the fluid collection device 100 at least proximate to the urethra of the user 150 when the first region 202 is disposed at least proximate to the supra-pubic region 152 of the user 150.

More specifically, the shape memory material 210 may extend longitudinally along the holder body 202 at least partially in the first region 204 and at least partially in the second region 206. The shape memory material 210 may be configured to bend and/or contour the holder body 202 to a substantially L-shaped configuration effective to dispose the first region 204 of the holder body 202 at least proximate to the supra-pubic region 152 of the user 150 while the second region 206 of the holder body 202 holds the opening 106 (not visible) of the fluid collection device 100 at least proximate to the urethra of the user 150. A user or caregiver may bend the holder 200 to conform or contour to the body of the user 150. The hole 214 is positioned and configured such that when the first region 204 is bent or adjusted relative to the second region 206, at least the conduit 108 extends through hole 214 rather than interfering with the first region 204.

In some embodiments, the first region 204 is configured to secure to the supra-pubic region 152 of the user 150. For example, the fluid collection assembly may include adhesive tape 410 configured to detachably secure the first region 204 to the supra-pubic region 152 of the user 150. In other embodiments, the first region may be secured to supra-pubic region of the user with an adhesive on the underside of the first region 204 of the holder body 202.

The fluid collection assembly 400 also may include an elbow connector 402 secured or securable to the conduit 108 that extends at least partially into the fluid collection device 100. In some embodiments, a flexible tube connector may be used in alternative or addition to the elbow connector 402. Flexible tube connectors are disclosed in PCT Patent Application No. PCT/US2022/015471, filed on Feb. 7, 2022, the disclosure of which is incorporated herein, in its entirety, by this reference. The elbow connector 402 (or the flexible tube connector) may be configured to secure to an additional conduit 408 effective to provide fluid communication between the conduit 108 and the additional conduit 408. The additional conduit 408 may be in fluid communication with a vacuum source such that fluid may be withdrawn from the fluid collection device 100 with the additional conduit 408 is in fluid communication with the conduit 108 disposed at least partially in the fluid collection device 100.

At least one, some, or all embodiments of fluid collection assembly 400 using anyone of the holders 200, 300, 350 disclosed herein are configured to maintain the correct and effective position of the fluid collection device 100 during use. At least one, some, or all embodiments of holders 200, 300, 350 used in the fluid collection assembly 400, then, result in the technical effect of more effectively collecting fluid from the user 150, preventing undesirable and embarrassing leaks of fluid, and/or improving the sanitary conditions of collected fluid from the user 150 by ensuring fluid is directed into the fluid collection device 100. At least one, some, or all embodiments of the fluid collection assembly 400 result in the technical effect of more simple and efficient changing of soiled or defective fluid collection devices 100. For example, during use, the holder 200 (or holder 300, 350) may not even need to be removed in order for a user or caregiver to change the fluid collection device being used.

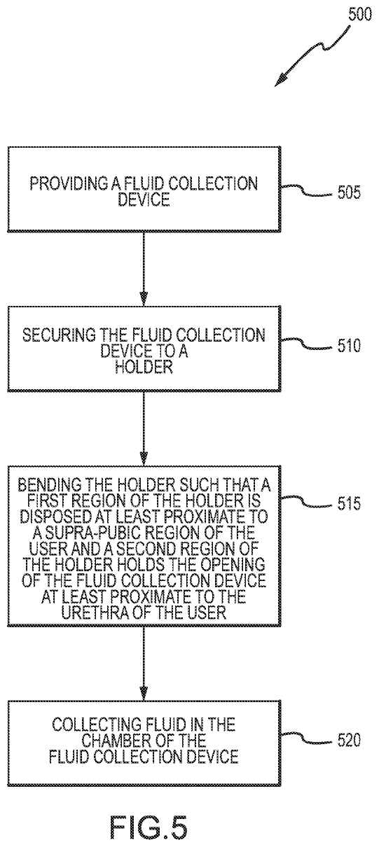

FIG. 5 is a flow diagram of a method 500 to collect fluid, according to an embodiment. The method 500 includes an act 505 of providing a fluid collection device having a chamber. The method 500 also includes an act 510 of securing the fluid collection device to a holder. The method 500 also may include an act 515 of bending the holder such that a first region of the holder is disposed at least proximate to a supra-pubic region of the user and a second region of the holder holds the opening of the fluid collection device at least proximate to the urethra of the user. The method 500 also may include an act 520 of collecting fluid in the chamber of the fluid collection device. In some embodiments, the method 500 may include an act of applying suction effective to suction the fluids from the chamber via a conduit disposed therein. Acts 505, 510, 515, 520 of the method 500 are for illustrative purposes. For example, the acts 505, 510, 515, 520 of the method 500 may be performed in different orders, split into multiple acts, modified, supplemented, or combined. Any of the acts 505, 510, 515, 520 may include using any of the fluid collection devices and/or holders disclosed herein.

The act 505 may include providing any of the fluid collection devices disclosed herein, such as the fluid collection device 100. For example, the act 505 may include providing a fluid collection device including a fluid impermeable barrier and a fluid permeable body. The fluid impermeable barrier of the fluid collection device provided in the act 505 may at least partially define a chamber, an opening extending longitudinally along the fluid impermeable barrier and configured to be positioned adjacent to a urethra of the user, and an aperture configured to receive a conduit therethrough. The fluid permeable body of the fluid collection device provided in the act 505 may be positioned at least partially within the chamber to extend across at least a portion of the opening and be configured to wick fluid away from the opening.

The act 510 of securing the fluid collection device to a holder may include securing the fluid collection device to any of the holders disclosed herein. In some embodiments, the act 510 includes inserting a distal end of the fluid impermeable barrier into a pocket on the second region of the holder body, the pocket being sized to hold the distal end of the fluid impermeable barrier therein without covering the opening of the fluid collection device. In some embodiments, the act 510 includes securing a strap on the second region of the holder body at least partially around a distal end of the fluid impermeable barrier to hold the distal end of the fluid impermeable barrier between the strap and the second region of the holder body without covering the opening of the fluid collection device.

In some embodiments, the method 500 further comprises inserting at least a conduit of the fluid collection device through a slot or a hole disposed at least partially in the first region of the holder. In some embodiments, the method 500 further comprises an act of inserting at least a conduit of the fluid collection device through a slot or a hole disposed at least partially in the first region of the holder body and between two of the at least longitudinal segments of the shape memory material. In some embodiments, the method 500 further comprises an act of inserting at least a conduit of the fluid collection device through a hole disposed at least partially in the first region of the holder body and between adjacent curves of the serpentine orientation or adjacent peaks of the zig-zag orientation of the shape memory material.

The act 515 of the method 500 may include bending the holder such that a first region of the holder is disposed at least proximate to a supra-pubic region of the user and a second region of the holder holds the opening of the fluid collection device at least proximate to the urethra of the user. In some embodiments, the act 515 includes bending shape memory material disposed in a holder body of the holder such that the holder body is substantially L-shaped with the first region of the holder disposed at least proximate to the supra-pubic region of the user and the second region of the holder holding the opening of the fluid collection device at least proximate to the urethra of the user. The shape memory material may extend longitudinally along the holder body at least partially in the first region and at least partially in the second region.

In some embodiments, the method 500 further comprises securing an elbow connector or a flexible connector to the conduit and securing the elbow connector or the flexible connector to an additional conduit effective to provide fluid communication between the conduit and the additional conduit. In some embodiments, the method 500 further comprises securing the first region to the supra-pubic region of the user. Securing the first region to the supra-pubic region of the user may include secure the first region to the supra-pubic region of the user with adhesive tape and/or an adhesive on the underside of the first region.

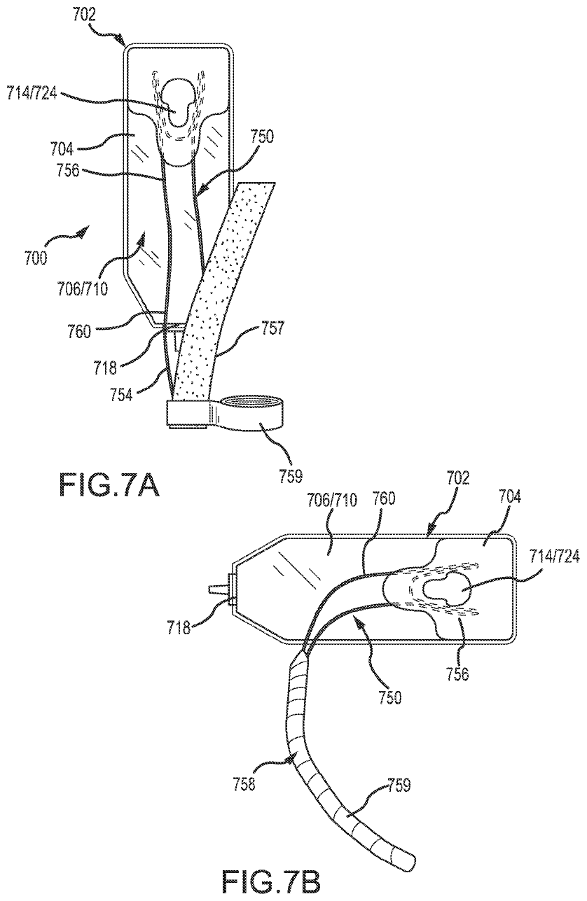

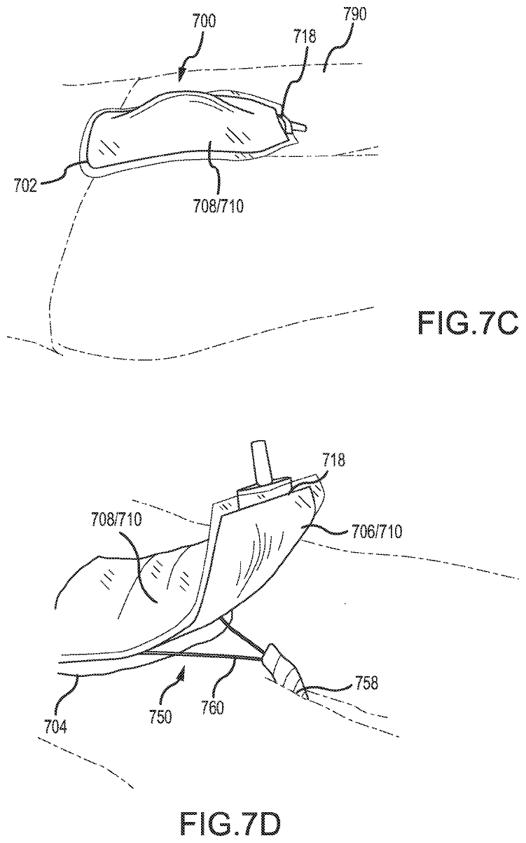

Holders disclosed here also may be used in fluid collection assemblies including a fluid collection device generally configured for use with male anatomy and/or used as for a urostomy. FIGS. 7A-7D, for example, illustrate a fluid collection assembly including a fluid collection device 700 and a holder 750 configured for use with male anatomy of a user 790. FIG. 7A is a partial disassembled view of the fluid collection assembly, FIG. 7B is an isometric view of the fluid collection assembly with a first region 704 of the holder 750 bent FIG. 7C is a front isometric view of the fluid collection assembly during use (the holder 750 is not visible), and FIG. 7D is an isometric view of the fluid collection assembly during use with the fluid collection device 700 lifted such that some of the holder 750 is visible.

The fluid collection device 700 includes a sheath 702 and a base 704, according to an embodiment. The sheath 702 includes a fluid impermeable barrier 706 that is at least partially formed from a first panel 708 attached to a second panel 710. In an embodiment, as illustrated, the first panel 708 and the second panel 710 are distinct sheets. The fluid impermeable barrier 706 also defines a chamber between the first panel 708 and the second panel 710, an opening 714, and an aperture 718 (e.g., fluid outlet). The sheath 702 also includes at least one porous material 722 disposed in the chamber. The base 704 includes an opening 724. The base 704 is attached to the proximal region of the fluid impermeable barrier 706 such that the opening 724 of the base 704 is aligned with the opening 714 in the fluid impermeable barrier 706. The inner surfaces of the fluid impermeable barrier 706 (e.g., inner surfaces of the first panel 708 and the second panel 710) at least partially defines the chamber within the fluid collection device 700. The fluid impermeable barrier 706 temporarily stores the bodily fluids in the chamber 712.

The fluid impermeable barrier 706 may be formed of any suitable fluid impermeable material(s), such as a fluid impermeable polymer (e.g., silicone, polypropylene, polyethylene, polyethylene terephthalate, polyurethane, polyethylene, polyvinyl chloride, a polycarbonate, etc.), a metal film, natural rubber, another suitable material, or combinations thereof. As such, the fluid impermeable barrier 706 substantially prevents the bodily fluids from passing through the fluid impermeable barrier 706. In an example, the fluid impermeable barrier 706 may be air permeable and fluid impermeable thus preventing leaks while allowing air flow through the chamber 712 when a vacuum force is applied thereto (i.e., the chamber 712 remains at about atmospheric pressure thereby preventing the vacuum force from causing a hickie or kinking the conduit). In such an example, the fluid impermeable barrier 706 may be formed of a hydrophobic material that defines a plurality of pores. At least one or more portions of at least an outer surface of the fluid impermeable barrier 706 may be formed from a soft and/or smooth material, thereby reducing chaffing.

In an embodiment, the fluid impermeable barrier 706 is formed from polyurethane, such as formed only from polyurethane. It has been found in clinical trials that forming the fluid impermeable barrier 706 from polyurethane improves the functionality of the fluid collection device 700. For example, the fluid impermeable barrier 706 may exhibit a flexibility when formed from polyurethane that is greater than when the fluid impermeable barrier 706 is formed from another material. The increased flexibility of the fluid impermeable barrier 706 formed from the polyurethane makes it easier to attach the fluid collection device 700 to the individual and maintain the fluid collection device 700 attached to the individual. The increased flexibility of the fluid impermeable barrier 706 formed from the polyurethane also help allow the fluid collection device 700 to conform to the individual and/or fit underneath the clothing of the individual thereby increasing patient comfort. The increased flexibility of the fluid impermeable barrier 706 prevents or at least inhibits corners formed in the fluid impermeable barrier 706, if present, from uncomfortably pressing into the individual using the fluid collection device 700 thereby making the fluid collection device 700 more comfortable to use. The polyurethane may also remain flexible when welded or otherwise have seals formed therein. It has also been found that individuals using the fluid collection device 700 describe the fluid impermeable barrier 706 formed from polyurethane as being more smooth than when the fluid impermeable barrier 706 is formed from other materials. The smoother feel of the fluid impermeable barrier 706 formed from polyurethane makes wearing the fluid collection device 700 more comfortable. It has also been found that forming the fluid impermeable barrier 706 from the polyurethane allows the fluid impermeable barrier 706 to be bent (e.g., crumpled, wrinkled, etc.) more quietly than if the fluid impermeable barrier 706 was formed from another material. The improved quietness of the fluid impermeable barrier 706 allows the fluid collection device 700 to be used more discreetly. For example, movement of an individual using the fluid collection device 700 is likely to cause the fluid impermeable barrier 706 to bend. Forming the fluid impermeable barrier 706 from polyurethane allows the individual to move more substantially without the fluid impermeable barrier 706 generating noise. Finally, it has been found that the fluid impermeable barrier 706 that includes polyurethane is able to be welded to a variety of materials thereby facilitating attachment of the base 704, the vents, and the port to the fluid impermeable barrier 706.

In an embodiment, at least one of the first panel 708 or the second panel 710 is formed from an at least partially transparent fluid impermeable material, such as polyethylene, polypropylene, polyurethane, polycarbonate, or polyvinyl chloride. Forming at least one of the first panel 708 or the second panel 710 from an at least partially transparent fluid impermeable material allows a person (e.g., medical practitioner) to examiner the penis. In some embodiments, both the first panel 708 and the second panel 710 are formed from at least partially transparent fluid impermeable material. For example, some conventional fluid collection assemblies that include a sheath and a base may allow the sheath to be reversibly detached from the base after the base is secured to the region about the penis. Detaching the sheath from the base allows the person to examine the penis. However, configuring the sheath to be detachable from the base may allow leaks between the sheath and the base. As previously discussed, the sheath 702 may be permanently attached to the base 704, which substantially prevents leaks between the sheath 702 and the base 704 when the base 704 is appropriately attached to the sheath 702 (e.g., no wrinkles were allowed to form between the sheath 702 and base 704). Selecting at least one of the first panel 708 or the second panel 710 to be formed from an at least partially transparent impermeable material allows the penis to be examined without detaching the entire fluid collection device 700 from the region about the penis. For example, the chamber 712 may include a penis receiving area 731 that is configured to receive the penis of the individual when the penis extends into the chamber 712. The penis receiving area 731 may be defined by at least the porous material 722 and at least a portion of the at least partially transparent material of the first panel 708 and/or the second panel 710. In other words, the porous material 722 is positioned in the chamber 712 such that the porous material is not positioned between the penis and at least a portion of the transparent portion of the first panel 708 and/or second panel 710 when the penis is inserted into the chamber 712 through the opening 714. The porous material 722 is generally not transparent and, thus, the portion of the at least partially transparent material of the first panel 708 and/or the second panel 710 that defines the penis receiving area 731 forms a window which allows the person to view into the penis receiving area 731 and examine the penis.

In an embodiment, the second panel 710 is at least partially formed from the at least partially transparent material and forms the window that allows the person to view into the penis receiving area. Further, the porous material 722 is positioned between the penis receiving area 731 and at least a portion of the first panel 708. Such an embodiment may help maintain the dignity of the individual using the fluid collection device 700. For example, during use, the second panel 710 is generally adjacent to the individual, such as adjacent to the thighs and/or perineum. Thus, the second panel 710 is generally obscured during use and a person cannot view the penis without first lifting the sheath 702 away from the individual. Meanwhile, the first panel 708 may face away from the individual and be more easily viewable than the second panel 710. However, a person (e.g., a passerby, a visitor, etc.) cannot view the penis through the first panel 708 because the porous material 722 is not transparent and/or the first panel 708 is formed from a non-transparent material. Thus, in such an embodiment, the first panel 708 and/or the porous material 722 prevent person(s) from viewing the penis unless such examination is necessary, thereby preserving the dignity of the individual using the fluid collection device 700. In an embodiment, the first panel 708 is formed from the at least partially transparent material and forms the window that allows the person to view into the penis receiving area. Further, the porous material 722 is positioned between the penis receiving area and at least a portion of the second panel 710. In such an embodiment, the person does not need to perform the additional act of lifting the sheath 702 to view into the penis receiving area but may not maintain the dignity of the individual using the fluid collection device 700 since passersby may also view into the penis receiving area.

As previously discussed, at least a portion of the first panel 708 and at least a portion of the second panel 710 are attached together. In an embodiment, as shown, the first and second panels 708, 710 are attached together along at least a portion of the outer edges thereof (e.g., the top and lateral edges). In such an embodiment, the first and second panels 708, 710 are attached using any suitable technique, such as with an adhesive, sewing, heat sealing, impulse heating, direct heating, radio frequency (“RF”) welding, ultrasonic (“US”) welding, or any other technique. In a particular embodiment, the first and second panels 708 are attached together using impulse heating since impulse heating is quick and is effective at attaching polyurethane panels together. Forming the fluid impermeable barrier 706 from the first panel 708 and the second panel 710 may improve the rate of manufacturing the fluid collection device 700, especially when the first panel 708 and the second panel 710 are attached together using a non-sewing technique.

In an embodiment, the fluid impermeable barrier 706 defines one or more orifices extending therethrough. For example, the sheath 702 may include one or more vents attached to the fluid impermeable barrier 706 that extend across the one or more orifices. The vents are configured to allow air to flow therethrough while preventing water (a major constituent of bodily fluids) from flowing therethrough. The vents may facilitate air flow through the chamber. For example, as previously discussed, a vacuum may be provided to the chamber from a vacuum source. The vacuum may pull air through the vents, thereby allowing air flow from the vents to the aperture 718. The air flow from the vents helps move bodily fluids towards the aperture 718. The vents also may prevent the vacuum applied to the chamber from bursting small superficial blood vessels (e.g., cause a hickie) in or otherwise damage the penis or the area about the penis. In some embodiments, the vents 734 may include a porous hydrophobic material. The pores defined by the porous hydrophobic material may be interconnected thereby allowing air to flow through the vents. The hydrophobic properties of the porous hydrophobic material may prevent water from flowing through the vents. In an example, the porous hydrophobic material may exhibit a contact angle with water that is greater than 90°. Generally, the size of the pores is dependent on the hydrophobicity of the porous hydrophobic material and vice versa. For example, increasing the hydrophobicity of the porous hydrophobic material allows the size of the pores to be increased.

In an embodiment, the vents may include a porous polytetrafluoroethylene (“PTFE”) layer. It is currently believed that the porous PTFE layer is exceptionally effectively at allowing air to flow therethrough while preventing water from flow therethrough even when the porous PTFE layer is exposed to acidic bodily fluids. However, porous PTFE is difficult to weld to other materials, such as polyurethane. As such, the porous PTFE layer may be attached to a substrate. The substrate may include a porous material or define one or more passageways therethrough. The substrate may be selected to be easily attached to the porous PTFE layer and the fluid impermeable barrier 706. For example, the substrate may be selected to be formed from polyvinyl chloride since polyvinyl chloride may be easily attached to the porous PTFE layer and attached to the fluid impermeable barrier 706.

The opening 714 defined by the fluid impermeable barrier 706 provides an ingress route for fluids to enter the chamber when the penis is a buried penis and allow the penis to enter the chamber (e.g., the penis receiving area) when the penis is not buried. The opening 714 may be defined by the fluid impermeable barrier 706 (e.g., an inner edge of the fluid impermeable barrier 706). For example, the opening 714 is formed in and extends through the fluid impermeable barrier 706, from the outer surface to the inner surface, thereby enabling bodily fluids to enter the chamber from outside of the fluid collection device 700.

In an embodiment, the second panel 710 defines the entirety of the opening 714. For example, the opening 714 is a cutout defined by the second panel 710 that is spaced from the outer periphery (e.g., edges) of the second panel 710. In such an example, the second panel 710 may exhibit a shape that substantially corresponds to the shape of the first panel 708 which may facilitate attaching the first panel 708 to the second panel 710 along the outer periphery thereof. It also allows the first panel 708 and the second panel 710 to lie substantially flat when the penis is not in the chamber 712 and the sheath 702 is lying on a flat surface. The ability of the first and second panels 708, 710 to lie substantially flat may make wearing the fluid collection device 700 more discrete and inhibit pooling of bodily fluids against the individual. However, in some embodiments, the opening 714 is not spaced from the outer periphery of the second panel 710. In such embodiments, the opening 714 may be a cutout extending inwardly from at least one outer periphery of the second panel 710. Other examples of forming the opening 714 in the second panel 710 are disclosed in International Patent Application No. PCT/US2021/039866 filed on Jun. 30, 2021, the disclosure of which is incorporated herein, in its entirety, by this reference.

As previously discussed, the fluid impermeable barrier 706 includes a proximal region and a distal region extending from the proximal region, for example, to the aperture 718 (e.g., to the port 730). The proximal region may define the opening 714 and the distal region may define the aperture 718. Both the proximal region and the distal region 762 may define the chamber.

In an embodiment, the fluid impermeable barrier 706 exhibits a generally bullet shape. As used, herein, the fluid impermeable barrier 706 exhibits the generally bullet shape when the proximal region 760 of the fluid impermeable barrier 706 exhibits a substantially constant first width and the distal region of the fluid impermeable barrier 706 exhibits a second width that is less than the first width. The first and second widths may be measured perpendicularly to the longitudinal axis and may be greater than a thickness of the sheath 702 when the sheath 702 lies on a flat surface. The generally bullet shape of the fluid impermeable barrier 706 facilitates operation of the fluid collection device 700 while making the sheath 702 more comfortable. For example, the substantially constant first width maintains contact with the upper portions of the thighs of the individual thereby maintaining the position of the fluid collection device 700. Also, the substantially constant first width makes it easier for the penis to become erect since the substantially constant first width inhibits the sheath 702 falling between the thighs of the individual and the erect penis from lifting the portions of the sheath 702 that fell between the thighs. The decreased second width makes the sheath 702 thinner thereby allowing the portions of the sheath 702 that are less likely to receive the penis to drop between the thighs of the individual. This drop allows gravity to assist in moving the bodily fluids towards the aperture 718. The decreased second width also funnels the bodily fluids towards the aperture 718 and the port. It is noted that the fluid impermeable barrier 706 may exhibit a shape other than the generally bullet shape. In an example, the fluid impermeable barrier 706 may exhibit a generally rectangular shape, as discussed in more detail in International Patent Application No. PCT/US2021/039866 filed on Jun. 30, 2021, the disclosure of which was previously incorporated herein. In an example, the fluid impermeable barrier 706 may exhibit a generally triangular shape, a semi-oval shape, or any other suitable shape.

The fluid impermeable barrier 706 defines the aperture 718, according to an embodiment. The fluid outlet 718 may be formed from a portion of the first panel 708 and the second panel 710 that are not attached together. In an embodiment, not shown, the aperture 718 is configured to be directly attached to a conduit. In such an embodiment, the conduit may be at least partially disposed in the chamber or otherwise in fluid communication with the chamber through the aperture 718. The aperture 718 may be sized and shaped to form an at least substantially fluid tight seal against the conduit, thereby substantially preventing the bodily fluids from escaping the chamber. The conduit may be attached to the aperture 718 (e.g., to the first and second panels 708, 710) using an adhesive, a weld, an interference or friction fit, or otherwise bonding the aperture 718 to the aperture 718. Attaching the conduit to the aperture 718 may prevent leaks and may prevent the conduit from inadvertently becoming detached from the aperture 718. In an example, the conduit may be attached to the aperture 718 in the same manufacturing step that attaches the first and second panels 708, 710 together. In an embodiment, as shown, the aperture 718 is configured to be indirectly attached to the conduit. In such an embodiment, the sheath 702 may include a port that is directly attached to the fluid impermeable barrier 706 and configured to be attached to the conduit.

Other examples and aspects of fluid collection devices or assemblies for use with males are disclosed in International Patent Application No. PCT/US2022/014285 filed on Jan. 28, 2022, the disclosure of which is incorporated herein, in its entirety, by this reference.

The holder 750 is secured or securable to the sheath 702 of the fluid collection device 700. In some embodiments, the holder 750 is selectively and/or detachably secured or securable to the sheath 702. In some embodiments, the sheath 702 is fixedly secured to the sheath 702. The holder 750 may include a shape memory material 760 including a first region 754 and a second region 756. In some embodiments, the holder 750 also may include a cover 758 covering at least some of the shape memory material 760. The shape memory material 760 of the holder 750 may include any material of other shape memory materials described or disclosed herein, such as the shape memory material 210. In some embodiments, the shape memory material 760 includes a first region 754 configured to be disposed between the legs of the user and bend to conform to a perineum of the user. The second region 756 of the shape memory material 760 is adjustable relative to the first region 754 and configured to hold the opening 714 of the fluid collection device 700 (e.g., the opening 724 of the base 704) against the user 790 with the penis extending at least partially into chamber of the fluid impermeable barrier through the opening 714, 724. In FIG. 7B, for example, the first region 754 of the shape memory material 760 is disposed inside a cover 758 and bent to a shape to conform to the perineum of the user. FIG. 7D illustrates an embodiment of the holder 750 in use with the cover 758 including the first region 754 of the shape memory material 760 bent to conform to the perineum of the user 790. This conforming of the first region 754 to the perineum of the user 790 assists in holding the base 704 against the user 790 with the penis of the user 790 extending through the opening 714, 724.

In some embodiments, at least some of the second region 756 of the shape memory material 760 is secured or securable to the sheath 702 between the base 704 and the second panel 710. For example, at least some of the second region 756 of the shape memory material 760 may be fixedly secured to the sheath 702 between the base 704 and the second panel 710 with an adhesive, sewing, heat sealing, impulse heating, direct heating, RF welding, US welding, or any other technique. In some embodiments, at least some of the second region 756 may be selective slid between the base 704 and the second panel 710 by the user 790 or caregiver. For example, multiple slots may be positioned between the base 704 and the second panel 710 and configured to receive at least some of the second region 756 of the shape memory material 760. With at least some of the second region 756 of the shape memory material 760 disposed between the base 704 and the second panel 710, the second region 756 of the shape memory material 760 may hold the base 704 against the user 790 with the penis of the user 790 extending through the opening 714, 724.

In some embodiments, at least a portion of shape memory material 760 of the second region 756 is positioned between the base 704 and the fluid impermeable barrier 706 (e.g., the second panel 710) and extends at least partially around the opening 724. For example, the second region 756 of the shape memory material 760 may be substantially Y-shaped. The Y-shape of the second region 756 may include two arms, and the opening 724 may be disposed between the two arms of the second region 756. In some embodiments, at least a portion of the shape memory material 760 of the second region 756 is positioned between the base 704 and the fluid impermeable barrier 710 (e.g., the second panel 110) and extends around all of the opening 724. For example, at least some of the second region 756 may form a circle or oval around the opening 724 between the base 704 and the second panel 110. In some embodiments, the shape memory material 760 of the second region includes two longitudinal segments extending longitudinally between the first region 754 and the opening 724 in the fluid impermeable barrier 710. The two longitudinal segments may be adjustable to fit testicles of the user 790 between the two longitudinal segments during use.

The cover 758 of the holder 750 may cover at least the shape memory material 760 of the first region 754. In some embodiments, the cover 758 includes a foam or padding material 757 disposed longitudinally at least partially around the shape memory material 760 of the first region 754 and a fluid impermeable layer 759 encasing the foam or padding material 757 and the shape memory material 760 of the first region 754. In some embodiments, the fluid impermeable layer 759 includes any material of the holder body 202 or the fluid impermeable barrier 102 encasing the foam or padding material 757 and the shape memory material 760 of the first region 754. In some embodiments, the foam or padding material 757 is absent, and the cover includes any material of the holder body 202 or the fluid impermeable barrier 102 encasing or covering the shape memory material 760 of the first region 754. In some embodiments, whether or not the holder 760 includes the foam or padding material 757, at least some (e.g., all) of the second region 756 of the shape memory material 760 may include a cover material including any material of the holder body 202 or the fluid impermeable barrier 102 encasing the shape memory material 760 of the second region 756.

A method of utilizing the fluid collection device 700 and holder 750 also is disclosed. In some embodiments, a method of collecting fluid from a user includes providing a fluid collection device 700 including a sheath 702 having a fluid impermeable barrier 706 having a proximal region and a distal region extending from the proximal region, an opening 724 in the proximal region, and an aperture 718 in the distal region. The fluid impermeable barrier 706 at least partially defines a chamber in fluid communication with the opening 724 and the aperture 718. The method also may include inserting a penis of the user through the opening 724 at least partially into the chamber and securing a base 704 of the fluid collection device 700 to the user at least proximate to the penis.

The method also may include bending a first region 754 of a holder 750 secured to the fluid collection device 700 such that the first region 754 of the holder 750 conforms to a perineum of the user between the legs of the user. The holder 750 may include a second region 756 secured or securable to the fluid collection device 700 at least proximate to the opening 724. In some embodiments, the method includes securing the second region 756 to the fluid collection device 700 between the base 704 and the fluid impermeable barrier 706. In some embodiments, bending the first region 754 of the holder 750 may include bending shape memory material 760 of the first region 754 of the holder such that the first region 754 conforms to the perineum of the user between the legs of the user.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiment disclosed herein are for purposes of illustration and are not intended to be limiting.

What is claimed is:

1. A fluid collection assembly, comprising:
a fluid collection device including a fluid impermeable barrier at least partially defining a chamber, the fluid impermeable barrier including an opening in fluid communication with the chamber, and an aperture substantially distal to the opening and in fluid communication with the chamber, the opening being configured to be positioned at least proximate to or receive therein a urethra of a user; and
a holder secured or securable to the fluid collection device, the holder including:
a first region configured to be disposed at least proximate to a supra-pubic region of the user or between the legs of the user; and
a second region adjustable relative to the first region and configured to hold the opening of the fluid collection device at least proximate to the urethra of the user when the first region is disposed at least proximate to the supra-pubic region of the user or against the user with the urethra received therein when the first region is disposed between the legs of the user.

2. The fluid collection assembly of claim 1, wherein:
the opening of the fluid collection device is configured to be placed at least proximate to the urethra of the user and the aperture is configured to receive a conduit therethrough to provide fluid communication between the chamber and the conduit;
the holder includes:
a holder body that is elongated and includes the first region and the second region; and
a shape memory material extending longitudinally along the holder body at least partially in the first region and at least partially in the second region, the shape memory material configured to bend and/or contour the holder body to a substantially L-shaped configuration effective to dispose the first region of the holder body at least proximate to the supra-pubic region of the user while the second region of the holder body holds the opening of the fluid collection device at least proximate to the urethra of the user.

3. The fluid collection assembly of claim 2, wherein the shape memory material includes at least two longitudinal segments extending longitudinally along the holder body at least partially in the first region and at least partially in the second region.

4. The fluid collection assembly of claim 3, wherein the shape memory material is formed or disposed as an elongated oval or square extending longitudinally along the holder body at least partially in the first region and at least partially in the second region and including the at least two longitudinal segments.

5. The fluid collection assembly of claim 3, wherein the shape memory material is formed or disposed as two or more elongated ovals or squares extending longitudinally along the holder body at least partially in the first region and at least partially in the second region and including the at least two longitudinal segments.

6. The fluid collection assembly of claim 3, wherein the shape memory material is formed or disposed to include the at least two longitudinal segments and a plurality of lateral segments extending between the at least two longitudinal segments.

7. The fluid collection assembly of any of claims 3-6, further comprising:
the conduit extending through the aperture of the fluid impermeable barrier of the fluid collection device; and
a slot or a hole disposed at least partially in the first region of the holder body and between two of the at least longitudinal segments of the shape memory material, the slot or the hole sized and dimensioned to receive at least the conduit therethrough.

8. The fluid collection assembly of claim 2, wherein the shape memory material is disposed in a serpentine orientation or a zig-zag orientation at least partially in the first region and at least partially in the second region.

9. The fluid collection assembly of claim 8, further comprising:
the conduit extending through the aperture of the fluid impermeable barrier of the fluid collection device; and
a hole disposed at least partially in the first region of the holder body and between adjacent curves of the serpentine orientation or adjacent peaks of the zig-zag orientation of the shape memory material, the hole sized and dimensioned to receive at least the conduit therethrough.

10. The fluid collection assembly of any of claims 2-8, wherein the holder further includes a pocket on the second region of the holder body, the pocket being sized to hold a distal end of the fluid impermeable barrier therein without covering the opening of the fluid collection device.

11. The fluid collection assembly of any of claims 2-8, wherein the holder further includes a strap positioned on the second region of the holder body to extend at least partially around a distal end of the fluid impermeable barrier and hold the distal end of the fluid impermeable barrier between the strap and the second region of the holder body without covering the opening of the fluid collection device.

12. The fluid collection assembly of claim 2, wherein the second region of the holder body includes a plurality of prongs configured to detachably secure the fluid collection device to the second region of the holder body, and the first region of the holder body includes a hole disposed at least partially therein, the hole sized and dimensioned to receive at least the conduit therethrough.

13. The fluid collection assembly of any of claims 2-12, wherein the holder body is molded to a shape generally complementary to the fluid collection device such that the holder body includes an elongated recess configured to receive at least a portion of the fluid impermeable barrier therein.

14. The fluid collection assembly of any of claims 2-13, wherein the holder body includes a fluid impermeable material.

15. The fluid collection assembly of claim 2, further comprising:
the conduit extending through the aperture of the fluid impermeable barrier of the fluid collection device; and
a slot or a hole disposed at least partially in the first region of the holder, the slot or the hole sized and dimensioned to receive at least the conduit therethrough.

16. The fluid collection assembly of any of claims 7, 9, 12, or 15, further comprising an elbow connector or a flexible connector secured or securable to the conduit and configured to secure to an additional conduit effective to provide fluid communication between the conduit and the additional conduit.

17. The fluid collection assembly of any of claims 2-16, wherein the first region is configured to secure to the supra-pubic region of the user.

18. The fluid collection assembly of claim 17, further comprising adhesive tape configured to secure the first region to the supra-pubic region of the user.

19. The fluid collection assembly of claim 1, wherein:
the fluid collection device includes a sheath including the fluid impermeable barrier having a proximal region including the opening and a distal region including the aperture, and a base secured or securable to the proximal region of the sheath and configured to be attached to the skin of the user around a penis of the user, wherein the opening is sized and dimension to receive at least a portion of the penis therethrough and the base extends at least partially around the opening; and
the holder includes a shape memory material including:
the first region configured be disposed between the legs of the user and bend to conform to a perineum of the user;
the second region adjustable relative to the first region and configured to hold the base of the fluid collection device against the user with the penis extending at least partially into the chamber of fluid impermeable barrier through the opening.

20. The fluid collection assembly of claim 19, wherein the first region includes a foam or padding material disposed longitudinally at least partially around the shape memory material and a fluid impermeable layer encasing the foam or padding material and the shape memory material of the first region.

21. The fluid collection assembly of any of claims 19 or 20, wherein at least a portion of shape memory material of the second region is positioned between the base and the fluid impermeable barrier and extends at least partially around the opening.

22. The fluid collection assembly of claim 21, wherein at least a portion of the shape memory material of the second region is positioned between the base and the fluid impermeable barrier and extends around all of the opening.

23. The fluid collection assembly of any of claims 19-21, wherein the shape memory material of the second region includes two longitudinal segments extending longitudinally between the first region of the holder and the opening in the fluid impermeable barrier, the two longitudinal segments being adjustable to fit testicles of the user between the two longitudinal segments during use.

24. A holder for a fluid collection device, the holder comprising:
a holder body that is elongated and includes a first region and a second region; and
a shape memory material extending longitudinally along the holder body at least partially in the first region and at least partially in the second region of the holder body, the shape memory material configured to bend and/or contour the holder body to a substantially L-shaped configuration effective to dispose the first region of the holder body at least proximate to a supra-pubic region of the user while the second region of the holder body holds an opening of the fluid collection device at least proximate to the urethra of the user.

25. The holder of claim 24, wherein the shape memory material includes at least two longitudinal segments extending longitudinally along the holder body at least partially in the first region and at least partially in the second region.

26. The holder of claim 25, wherein the shape memory material is formed or disposed as an elongated oval or square extending longitudinally along the holder body at least partially in the first region and at least partially in the second region and including the at least two longitudinal segments.

27. The holder of claim 25, wherein the shape memory material is formed or disposed as two or more elongated ovals or squares extending longitudinally along the holder body at least partially in the first region and at least partially in the second region and including the at least two longitudinal segments.

28. The holder of claim 25, wherein the shape memory material is formed or disposed to include the at least two longitudinal segments and a plurality of lateral segments extending between the at least two longitudinal segments.

29. The holder of any of claims 25-28, further comprising a slot or a hole disposed at least partially in the first region of the holder body and between two of the at least longitudinal segments of the shape memory material, the slot or the hole sized and dimensioned to receive at least a conduit of the fluid collection device therethrough.

30. The holder of claim 24, wherein the shape memory material is disposed in a serpentine orientation or a zig-zag orientation at least partially in the first region and at least partially in the second region.

31. The holder of claim 30, further comprising a hole disposed at least partially in the first region of the holder body and between adjacent curves of the serpentine orientation or adjacent peaks of the zig-zag orientation of the shape memory material, the hole sized and dimensioned to receive at least a conduit of the fluid collection device therethrough.

32. The holder of any of claims 24-31, further comprising a pocket on the second region of the holder body, the pocket being sized to hold a distal end of the fluid collection device.

33. The holder of any of claims 24-31, wherein the holder further comprises a strap positioned on the second region of the holder body to extend at least partially around a distal end of the fluid collection device and hold the distal end of the fluid collection device between the strap and the second region of the holder body.

34. The holder of claim 24, wherein the second region of the holder body includes a plurality of prongs configured to detachably secure the fluid collection device to the second region of the holder body, and the first region of the holder body includes a hole disposed at least partially therein, the hole sized and dimensioned to receive at least the conduit therethrough.

35. The holder of any of claims 24-34, wherein the holder body is molded to a shape generally complementary to the fluid collection device such that the holder body includes an elongated recess configured to receive at least a portion of the fluid collection device therein.

36. The holder of any of claims 24-34, wherein the holder body includes a fluid impermeable material.

37. The holder of any of claims 24-36, wherein the first region is configured to secure to the supra-pubic region of the user with adhesive tape.

38. A method of collecting fluid from a user, the method comprising:
providing a fluid collection device including a fluid impermeable barrier defining a chamber, an opening having fluid communication with the chamber and configured to be placed proximate to a urethra of the user, and an aperture configured to receive a conduit therethrough to provide fluid communication between the chamber and the conduit;
securing the fluid collection device to a holder;
bending the holder such that a first region of the holder is disposed at least proximate to a supra-pubic region of the user and a second region of the holder holds the opening of the fluid collection device at least proximate to the urethra of the user; and
collecting fluid in the chamber of the fluid collection device.

39. The method of claim 38, wherein bending the holder includes bending shape memory material disposed in a holder body of the holder such that the holder body is substantially L-shaped with the first region of the holder disposed at least proximate to the supra-pubic region of the user and the second region of the holder holding the opening of the fluid collection device at least proximate to the urethra of the user, the shape memory material extending longitudinally along the holder body at least partially in the first region and at least partially in the second region.

40. The method of claim 39, wherein the shape memory material includes at least two longitudinal segments extending longitudinally along the holder body at least partially in the first region and at least partially in the second region.

41. The method of claim 40, wherein the shape memory material is formed or disposed as an elongated oval or square extending longitudinally along the holder body at least partially in the first region and at least partially in the second region and including the at least two longitudinal segments.

42. The method of claim 40, wherein the shape memory material is formed or disposed as two or more elongated ovals or squares extending longitudinally along the holder body at least partially in the first region and at least partially in the second region and including the at least two longitudinal segments.

43. The method of any of claims 40-42, further comprising inserting at least a conduit of the fluid collection device through a slot or a hole disposed at least partially in the first region of the holder body and between two of the at least longitudinal segments of the shape memory material.

44. The method of claim 39, wherein the shape memory material is disposed in a serpentine orientation or a zig-zag orientation at least partially in the first region and at least partially in the second region.

45. The method of claim 44, further comprising inserting at least a conduit of the fluid collection device through a hole disposed at least partially in the first region of the holder body and between adjacent curves of the serpentine orientation or adjacent peaks of the zig-zag orientation of the shape memory material.

46. The method of any of claims 39-45, wherein securing the fluid collection device to the holder includes inserting a distal end of the fluid impermeable barrier into a pocket on the second region of the holder body, the pocket being sized to hold the distal end of the fluid impermeable barrier therein without covering the opening of the fluid collection device.

47. The method of any of claims 39-45, wherein securing the fluid collection device to the holder includes securing a strap on the second region of the holder body at least partially around a distal end of the fluid impermeable barrier to hold the distal end of the fluid impermeable barrier between the strap and the second region of the holder body without covering the opening of the fluid collection device.

48. The method of any of claims 39-47, wherein the holder body includes a fluid impermeable material.

49. The method of any of claims 38 or 39, further comprising inserting at least a conduit of the fluid collection device through a slot or a hole disposed at least partially in the first region of the holder.

50. The method of any of claims 43, 45, or 49, further comprising securing an elbow connector or a flexible connector to the conduit and securing the elbow connector or the flexible connector to an additional conduit effective to provide fluid communication between the conduit and the additional conduit.

51. The method of any of claims 38-50, further comprising securing the first region to the supra-pubic region of the user.

52. The method of claim 38-50, further comprising securing the first region to the supra-pubic region of the user with adhesive tape.

53. A method of collecting fluid from a user, the method comprising:
providing a fluid collection device including a sheath having a fluid impermeable barrier having a proximal region and a distal region extending from the proximal region, an opening in the proximal region, and an aperture in the distal region, the fluid impermeable barrier at least partially defining a chamber in fluid communication with the opening and the aperture;
inserting a penis of the user through the opening at least partially into the chamber;
securing a base of the fluid collection device to the user at least proximate to the penis;
bending a first region of a holder secured to the fluid collection device such that the first region of the holder conforms to a perineum of the user between the legs of the user, the holder including a second region secured to the fluid collection device at least proximate to the opening.

54. The method of claim 53, wherein bending the first region of the holder includes bending shape memory material of the first region of the holder such that the first region of the holder conforms to the perineum of the user between the legs of the user.

55. The method of any of claims 53 or 54, wherein the second region of the holder includes shape memory material extending at least partially around the opening.

56. A holder for a fluid collection device, the holder comprising:
a first region that is elongated and including a shape memory material configured to bend to conform to a perineum between legs of a user; and
a second region extending from the first region and configured to secure to a fluid collection device proximate to an opening in the fluid collection device for insertion of the a penis, the second region being adjustable relative to the first region and, when secured to the fluid collection device, configured to hold the opening of the fluid collection device against the user when the penis of the user is inserted into the opening.

57. The holder of claim 56, wherein the first region includes a foam or padding material disposed longitudinally at least partially around the shape memory material and a fluid impermeable layer encasing the foam or padding material and the shape memory material of the first region.

58. The holder of any of claims 56 or 57, wherein the second region includes shape memory material configured to secured to the fluid collection device at least partially around the opening of the fluid collection device.

59. The holder of claim 58, wherein the second region is substantially Y-shaped with two arms disposed between the base and the fluid impermeable barrier, the opening between disposed between the two arms of the second region.

60. The holder of claim 58, wherein the shape memory material of the second region is configured to extend around all of the opening of the fluid collection device.

61. The holder of any of claims 58-60, wherein the shape memory material of the second region includes two longitudinal segments extending longitudinally between the first region of the holder, the two longitudinal segments being adjustable to fit testicles of the user between the two longitudinal segments during use.
